(19) United States
(12) Patent Application Publication
(10) Pub. No.: US 2010/0331742 A1
(43) Pub. Date: Dec. 30, 2010

(54) SURGICAL OPERATING APPARATUS

(76) Inventor: Shinya MASUDA, Hino-shi (JP)

Correspondence Address: OSTROLENK FABER GERB & SOFFEN, 1180 AVENUE OF THE AMERICAS, NEW YORK, NY, 100368403, US

(21) Appl. No.: 12/492,268

(22) Filed: Jun. 26, 2009

(51) Int. Cl. A61B 18/14; A61N 7/00

(52) U.S. Cl. 601/2; 606/42

(57) ABSTRACT

A surgical operating apparatus includes a probe for outputting energy for performing therapeutic treatment of a living body, a jaw which is openable/closable relative to the probe, and an output mode switching section which selectively switches, in accordance with an open/closed state of the jaw, an output of the surgical operating apparatus between a bipolar mode in which the probe and the jaw are used as high-frequency electrodes, and a probe-only output mode in which the energy is output from only the probe.

### BACKGROUND OF THE INVENTION

The present invention relates to a surgical operating apparatus which performs therapeutic treatment, such as incision, resection or coagulation, of a living body tissue.

Jpn. Pat. Appln. KOKAI Publication No. 2005-237574 (Patent Document 1), for instance, discloses a high-frequency therapeutic apparatus as a general example of a surgical operating apparatus which can perform therapeutic treatment, such as incision, resection or coagulation, of a living body tissue by making use of a high-frequency current.

In this apparatus, a proximal-side operation section is coupled to a proximal end portion of an elongated insertion section. An electric cord for supplying high-frequency current from a high-frequency cauterization power supply device is connected to the operation section. A therapeutic section for treating a living body tissue is provided at a distal end portion of the insertion section.

The therapeutic section is provided with a pair of jaws. An operation rod for driving the jaws is inserted in a sheath so as to be axially advancible/retreatable. Further, the high-frequency cauterization power supply device is electrically connected to the jaws of the therapeutic section via an electric path in the operation section and the sheath.

In accordance with the operation of the operation section, the operation rod is axially advanced/retreated. In interlock with the operation of the operation rod, the jaws are opened/closed. At this time, a living body tissue is held between the paired jaws in accordance with the closing operation of the jaws. In this state, a high-frequency current is supplied to the jaws of the therapeutic section, and high-frequency therapeutic treatment, such as coagulation, of the living body tissue is performed.

High-frequency therapeutic devices are classified into devices of a so-called monopolar type and devices of a so-called bipolar type. In the therapeutic device of the monopolar type, when high-frequency therapeutic treatment is performed, a counter-electrode plate is disposed on the outside of the patient's body. When the high-frequency therapeutic treatment is performed, a high-frequency current is let to flow from the therapeutic device to the counter-electrode plate via a living body tissue. The monopolar type therapeutic device, in many cases, is preferably used when a membranous tissue with a low risk of bleeding, for instance, is treated quickly.

In the bipolar type therapeutic device, a treatment section at a distal end portion of the insertion section is provided with a pair of electrically insulated electrodes. A high-frequency current is let to flow between the two electrodes in the state in which the paired electrodes are put in contact with a living tissue at the same time. Thereby, high-frequency heating is performed on the living tissue. In many cases, the bipolar type therapeutic device is used for the purpose of hemostasis of a region which tends to easily bleed, or a region which is bleeding. The above-described Patent Document 1 discloses an example of the bipolar therapeutic device.

### BRIEF SUMMARY OF THE INVENTION

According to a first aspect of the present invention, a surgical operating apparatus comprising: a probe for outputting energy for performing therapeutic treatment of a living body; a jaw which is openable/closable relative to the probe; and an output mode switching section which selectively switches, in accordance with an open/closed state of the jaw, an output of the surgical operating apparatus between a bipolar mode in which the probe and the jaw are used as high-frequency electrodes, and a probe-only output mode in which the energy is output from only the probe.

According to another aspect of the present invention, there is provided a surgical operating apparatus comprising: a probe for outputting energy for performing therapeutic treatment of a living body; a jaw which is openable/closable relative to the probe; an operation section for opening/closing the jaw; and an output mode switching section which selectively switches an output mode of the probe between a probe-only output mode in which the energy is output from only the probe, and a bipolar mode in which the probe and the jaw are used as high-frequency electrodes, wherein the output mode switching section includes a contact-point switching section which opens/closes a contact point that switches on/off an electrical conduction state of a high-frequency current to the jaw in accordance with an operation of a movable member which moves in accordance with an operation of the operation section.

According to another aspect of the present invention, there is provided a surgical operating apparatus comprising: a probe for outputting energy for performing therapeutic treatment of a living body; a jaw which is openable/closable relative to the probe; and output mode switching means for effecting switching between a bipolar mode in which the probe and the jaw are used as electrodes, and a probe-only output mode in which energy is output from only the probe, wherein the output mode switching means operates in interlock with an opening/closing operation of the jaw, effects switching to the bipolar mode at a time of a closing operation of the jaw, and effects switching to the probe-only output mode at a time of an openable operation of the jaw.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view that schematically shows the entire structure of an ultrasonic therapeutic apparatus according to a first embodiment of the present invention;

FIG. 2 is a perspective view showing a state in which coupling sections of the ultrasonic therapeutic apparatus according to the first embodiment are disconnected;

FIG. 3A is a plan view showing a distal end portion of a sheath unit of the ultrasonic therapeutic apparatus according to the first embodiment;

FIG. 3B is a plan view showing a distal end portion of a probe unit of the ultrasonic therapeutic apparatus according to the first embodiment;

FIG. 4A is a longitudinal cross-sectional view showing a distal end portion of the sheath unit of the ultrasonic therapeutic apparatus according to the first embodiment;

FIG. 4B is a longitudinal cross-sectional view showing an insulating coating of an inner peripheral surface of an inner cylinder;

FIG. 5 is a cross-sectional view taken along line V-V in FIG. 4A;

FIG. 6 is a cross-sectional view taken along line VI-VI in FIG. 4A;

FIG. 7 is a cross-sectional view taken along line VII-VII in FIG. 4A;

FIG. 8 is a longitudinal cross-sectional view showing a proximal end portion of the sheath unit of the ultrasonic therapeutic apparatus according to the first embodiment;

FIG. 9A is a cross-sectional view taken along line IXA-IXA in FIG. 8;

FIG. 9B is a cross-sectional view taken along line IXB-IXB in FIG. 8;

FIG. 10 is a cross-sectional view taken along line X-X in FIG. 8;

FIG. 11 is a cross-sectional view taken along line XI-XI in FIG. 8;

FIG. 12 is a perspective view showing a connection tube body of the sheath unit of the ultrasonic therapeutic apparatus according to the first embodiment;

FIG. 13 is a side view showing the connection tube body of the sheath unit of the ultrasonic therapeutic apparatus according to the first embodiment;

FIG. 14 is a side view showing a coupled state between a handle unit and a transducer unit of the ultrasonic therapeutic apparatus according to the first embodiment;

FIG. 15 is a longitudinal cross-sectional view showing a unit coupling portion of the ultrasonic therapeutic apparatus according to the first embodiment;

FIG. 16 is a longitudinal cross-sectional view showing an internal structure of the handle unit of the ultrasonic therapeutic apparatus according to the first embodiment;

FIG. 17A is a cross-sectional view, taken along line 17-17 in FIG. 16, showing a state before engagement between the handle unit and the sheath unit of the ultrasonic therapeutic apparatus according to the first embodiment;

FIG. 17B is a cross-sectional view, taken along line 17-17 in FIG. 16, showing a state after engagement between the handle unit and the sheath unit of the ultrasonic therapeutic apparatus according to the first embodiment;

FIG. 18 is a cross-sectional view taken along line 18-18 in FIG. 16;

FIG. 19 is a cross-sectional view taken along line 19-19 in FIG. 16;

FIG. 20 is a cross-sectional view taken along line 20-20 in FIG. 16;

FIG. 21 is a cross-sectional view taken along line 21-21 in FIG. 16;

FIG. 22 is a cross-sectional view taken along line 22-22 in FIG. 16;

FIG. 23 is a cross-sectional view taken along line 23-23 in FIG. 16;

FIG. 24 is a cross-sectional view taken along line 24-24 in FIG. 16;

FIG. 25 is a cross-sectional view taken along line 25-25 in FIG. 16;

FIG. 26 is a perspective view showing an electrode hold member of the ultrasonic therapeutic apparatus according to the first embodiment;

FIG. 27 is a front view showing the electrode hold member of the ultrasonic therapeutic apparatus according to the first embodiment;

FIG. 28 is a side view showing the electrode hold member of the ultrasonic therapeutic apparatus according to the first embodiment;

FIG. 29 is a perspective view showing an electrode member of the ultrasonic therapeutic apparatus according to the first embodiment;

FIG. 30 is a transverse cross-sectional view showing the electrode member of the ultrasonic therapeutic apparatus according to the first embodiment;

FIG. 31 is a perspective view showing a state before the rotational engagement at the time when the handle unit and sheath unit of the ultrasonic therapeutic apparatus according to the first embodiment are coupled;

FIG. 32 is a plan view showing a state before the rotational engagement at the time when the handle unit and sheath unit of the ultrasonic therapeutic apparatus according to the first embodiment are coupled;

FIG. 33 is a perspective view showing a state after the rotational engagement at the time when the handle unit and sheath unit of the ultrasonic therapeutic apparatus according to the first embodiment are coupled;

FIG. 34 is a plan view showing a state after the rotational engagement at the time when the handle unit and sheath unit of the ultrasonic therapeutic apparatus according to the first embodiment are coupled;

FIG. 35 is a side view showing a state before an attachment member is assembled to a base member of a stationary handle of the handle unit of the ultrasonic therapeutic apparatus according to the first embodiment;

FIG. 36 is a plan view showing the probe unit of the ultrasonic therapeutic apparatus according to the first embodiment;

FIG. 37 is a cross-sectional view taken along line 37-37 in FIG. 36;

FIG. 38 is a plan view showing a coupled state between the transducer unit and a cable of the ultrasonic therapeutic apparatus according to the first embodiment;

FIG. 39 is a plan view showing a proximal end portion of the transducer unit and the cable of the ultrasonic therapeutic apparatus according to the first embodiment;

FIG. 40 is a schematic view showing the structure of an electric path of the transducer unit of the ultrasonic therapeutic apparatus according to the first embodiment;

FIG. 41 is a longitudinal cross-sectional view showing an internal structure of a front end portion of the transducer unit of the ultrasonic therapeutic apparatus according to the first embodiment;

FIG. 42 is a longitudinal cross-sectional view of a main part, showing a state in which an output mode switching section of the ultrasonic therapeutic apparatus according to the first embodiment is switched to a probe-only output mode;

FIG. 43 is a longitudinal cross-sectional view of a main part, showing a state in which the ultrasonic therapeutic apparatus according to the first embodiment is switched to a bipolar mode;

FIG. 44 schematically shows the structure of an electric circuit of the entire system of the ultrasonic therapeutic apparatus according to the first embodiment;

FIG. 45 is a side view of a main part, showing a state in which a handpiece of the ultrasonic therapeutic apparatus according to the first embodiment is used as a monopolar therapeutic apparatus;

FIG. 46 is a longitudinal cross-sectional view showing an internal structure of a handpiece of an ultrasonic therapeutic apparatus according to a second embodiment of the present invention;

FIG. 47 is a longitudinal cross-sectional view of a main part, showing a state in which an output mode switching section of the handpiece of the ultrasonic therapeutic apparatus according to the second embodiment is switched to a probe-only output mode;

FIG. 48 is a longitudinal cross-sectional view of a main part, showing a state in which the output mode switching section of the handpiece of the ultrasonic therapeutic apparatus according to the second embodiment is switched to a bipolar mode;

FIG. 49 is a longitudinal cross-sectional view showing an internal structure of a handpiece of an ultrasonic therapeutic apparatus according to a third embodiment of the present invention;

FIG. 50 is a longitudinal cross-sectional view of a main part, showing a state in which an output mode switching section of the handpiece of the ultrasonic therapeutic apparatus according to the third embodiment is switched to a probe-only output mode;

FIG. 51 is a longitudinal cross-sectional view of a main part, showing a state in which the output mode switching section of the handpiece of the ultrasonic therapeutic apparatus according to the third embodiment is switched to a bipolar mode;

FIG. 52 schematically shows the structure of an electric circuit of the entire system of a surgical operating apparatus according to a fourth embodiment of the invention;

FIG. 53 schematically shows the structure of an electric circuit of the entire system of a surgical operating apparatus according to a fifth embodiment of the invention;

FIG. 54 schematically shows the structure of an electric circuit of the entire system of a surgical operating apparatus according to a sixth embodiment of the invention;

FIG. 55 schematically shows the structure of an electric circuit of the entire system of a surgical operating apparatus according to a seventh embodiment of the invention; and

FIG. 56 schematically shows the structure of an electric circuit of the entire system of a surgical operating apparatus according to an eighth embodiment of the invention.

### DETAILED DESCRIPTION OF THE INVENTION

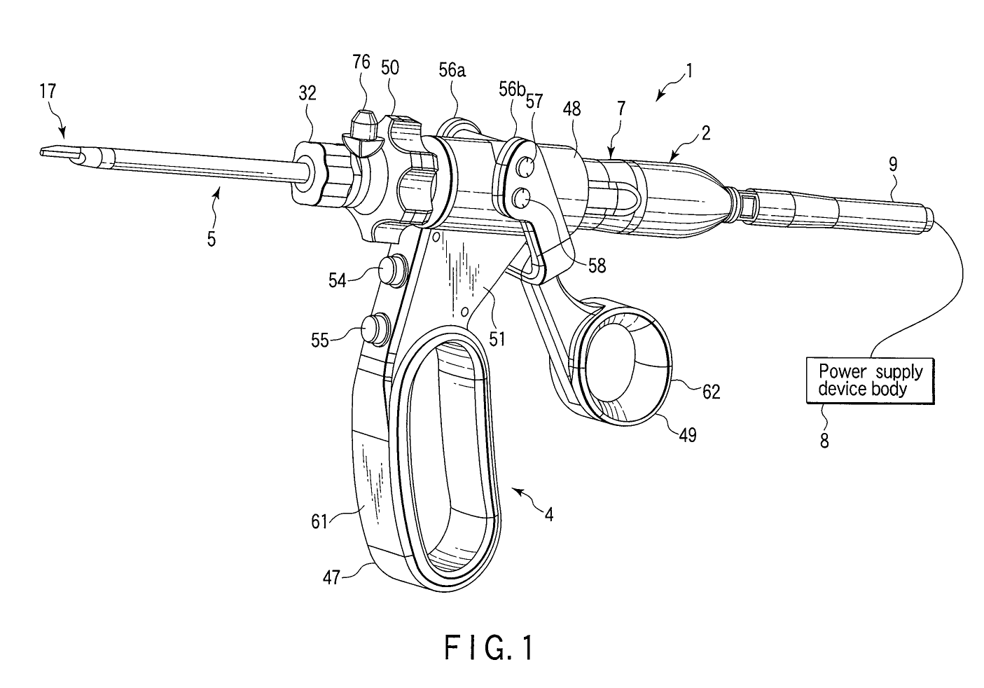

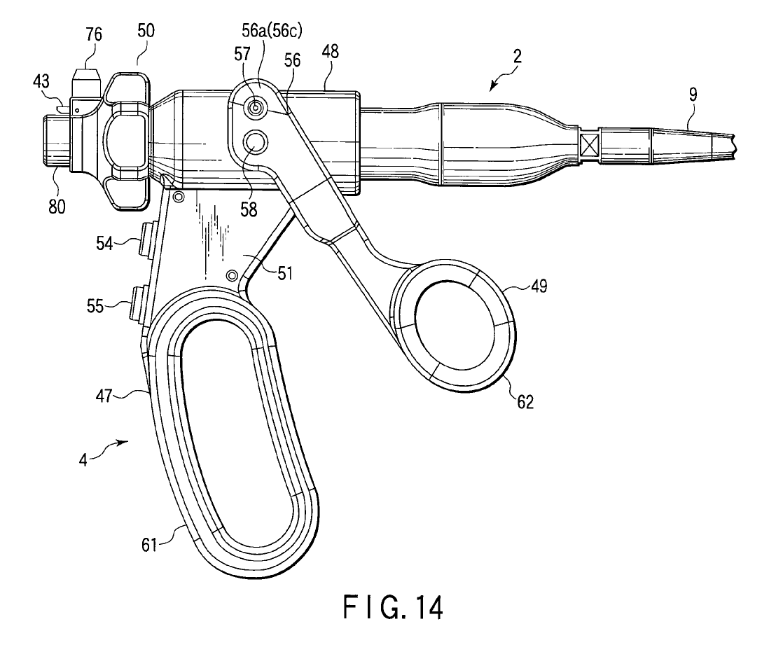

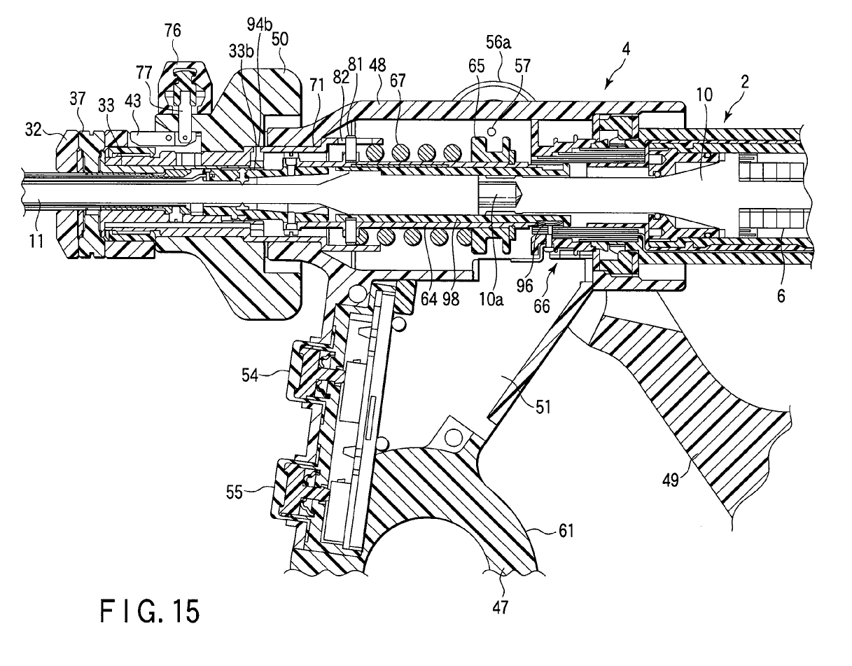

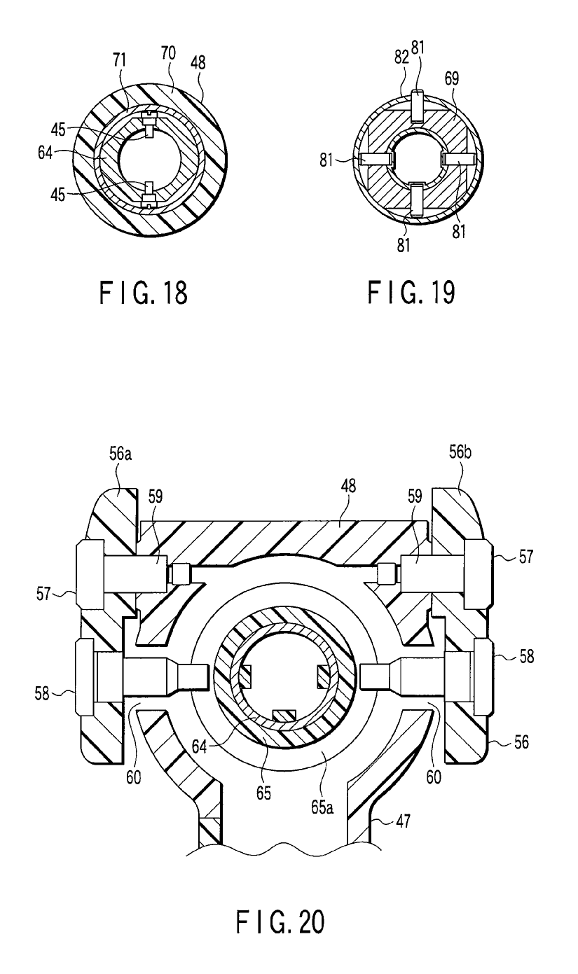

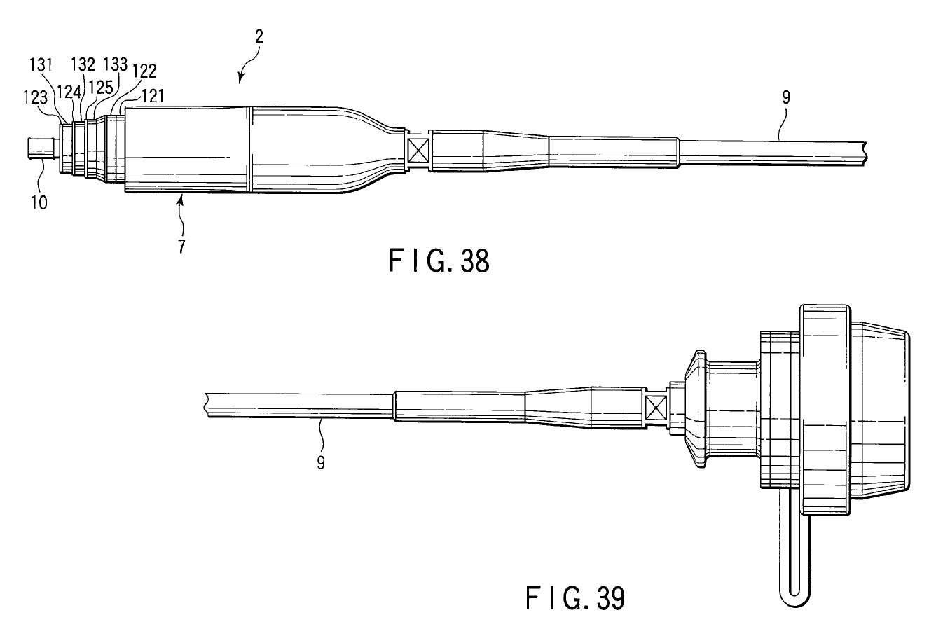

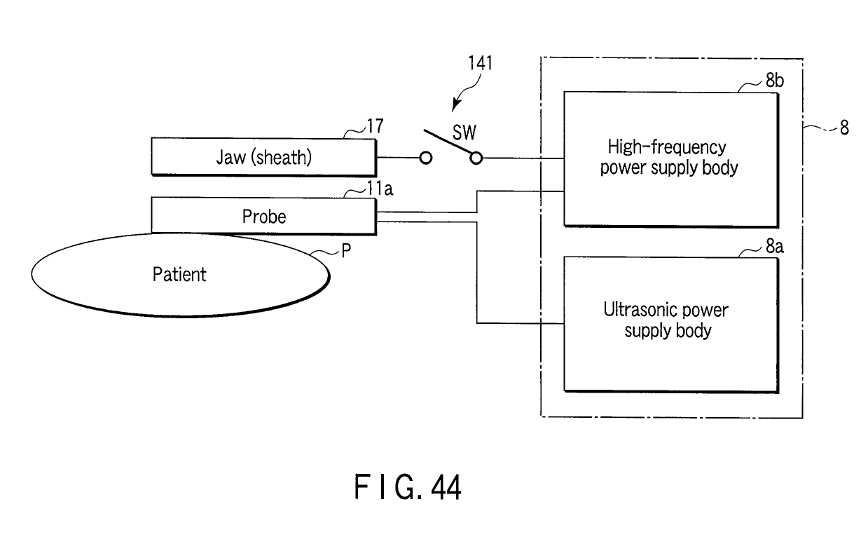

A first embodiment of the present invention will now be described with reference to FIG. 1 to FIG. 45. FIG. 1 schematically shows the entire structure of a handpiece 1 of an ultrasonic therapeutic apparatus which is a surgical operating apparatus according to the present embodiment. The ultrasonic therapeutic apparatus of the present embodiment is an ultrasonic coagulation/incision therapeutic apparatus which can perform therapeutic treatment, such as incision, resection or coagulation, of a living body tissue by making use of ultrasonic, and can also perform therapeutic treatment by high-frequency waves. FIG. 44 schematically shows the structure of an electric circuit of the entire system of the ultrasonic therapeutic apparatus.

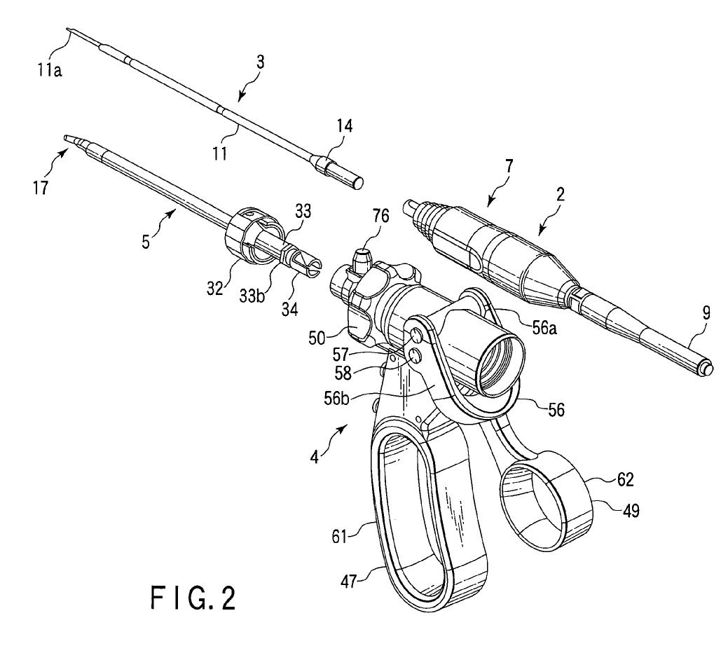

The handpiece 1, as shown in FIG. 2, comprises four units, namely, a transducer unit (ultrasonic output section) 2, a probe unit (probe section) 3, a handle unit (handle section) 4 and a sheath unit (sheath section) 5. These four units are detachably coupled.

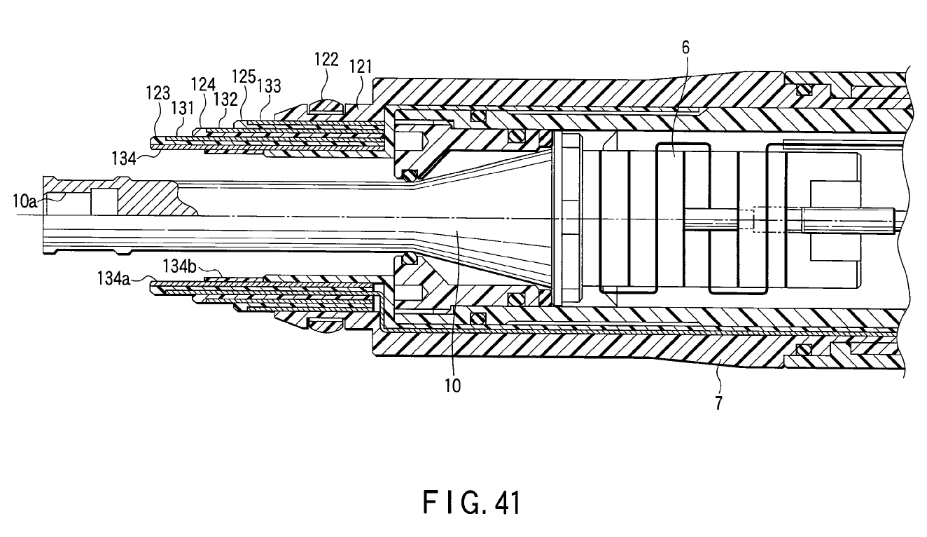

A transducer 6 (see FIG. 41), which will be described later, is assembled in the transducer unit 2. The transducer 6 generates ultrasonic vibration by a piezoelectric element which converts an electric current to ultrasonic vibration. An outside of the piezoelectric element is covered with a circular cylindrical transducer cover 7. Further, one end of a cable 9 is connected to a rear end of the transducer unit 2. The other end of the cable 9 is connected to a power supply device body 8. As shown in FIG. 44, the power supply device body 8 includes an ultrasonic power supply body 8a for supplying an electric current for generating ultrasonic vibration, and a high-frequency power supply body 8b for supplying a high-frequency current.

A proximal end portion of a horn 10, which increases the amplitude of ultrasonic vibration, is coupled to a front end portion of the ultrasonic transducer 6 within the transducer cover 7. A screw hole portion 10a for attaching the probe is formed at a distal end portion of the horn 10.

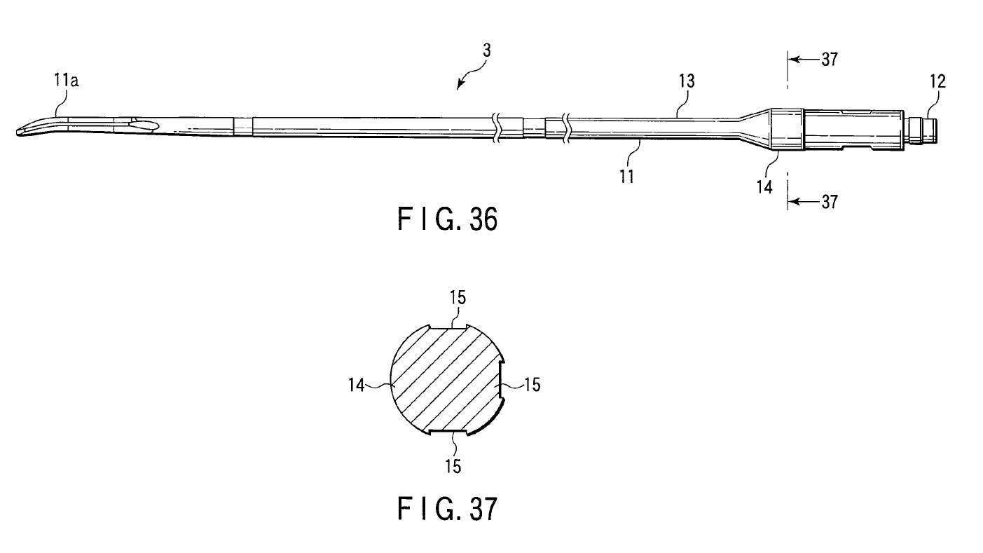

FIG. 36 shows the external appearance of the entire probe unit 3. The probe unit 3 is designed such that the entire length thereof may become an integer number of times of half-wave length of the ultrasonic vibration. The probe unit 3 includes a metallic rod-shaped probe (vibration transmission member) 11. A proximal end portion of the probe 11 is provided with a screw portion 12 which is to be engaged with the screw hole portion 10a of the horn 10. The screw portion 12 is engaged with the screw hole portion 10a of the horn 10 of the transducer unit 2. Thereby, the probe unit 3 and the transducer unit 2 are assembled. At this time, a first high-frequency electric path 13, through which a high-frequency current is transmitted, is formed in the coupled body of the ultrasonic transducer 6 and the probe unit 3.

A probe distal end portion 11a is provided at a distal end portion of the probe 11. The probe distal end portion 11a is formed in a substantially J-shaped curved form. The cross-sectional area of the probe unit 3 is decreased in the axial direction at several nodes of vibration in the axial direction, so that an amplitude necessary for therapeutic treatment can be obtained at the probe distal end portion 11a. Rubber rings, which are formed of elastic material in an annular shape, are attached to several positions of nodes of vibration along the axial direction of the probe unit 3. These rubber rings prevent interference between the probe unit 3 and the sheath unit 5.

A flange portion 14 is provided at the position of the node of vibration on the most proximal end side in the axial direction of the probe unit 3. As shown in FIG. 37, engaging recess portions 15 each having a key groove shape are formed on the outer peripheral surface of the flange portion 14 at three positions in the circumferential direction thereof.

The sheath unit 5 includes a sheath body 16, which is formed of a circular cylindrical body, and a jaw 17 which is provided at a distal end of the sheath body 16. The sheath body 16, as shown in FIG. 7, includes a metallic outer cylinder 18 having a circular cross-sectional shape, and a metallic inner cylinder 19 having a non-circular cross-sectional shape, for example, a D-shaped cross section. A channel 22 for passing a driving shaft 21 of the jaw 17 is formed between the outer cylinder 18 and the inner cylinder 19.

As shown in FIG. 4A, the outer peripheral surface of the outer cylinder 18 is covered with an insulation tube 23. As shown in FIG. 4B, the inner peripheral surface of the inner cylinder 19 is provided with an insulation coating 24 of an insulating material. An insulation tube may be provided on the inner peripheral surface of the inner cylinder 19. Electrical insulation from the probe unit 3 is ensured by the insulation coating 24 on the inner cylinder 19.

A proximal end portion of a substantially circular cylindrical distal end cover 25 is fixed to a distal end portion of the outer cylinder 18. A pipe-shaped hold member 26, which holds the probe unit 3 so as not to come in contact with the distal end cover 25, is attached to an inner peripheral surface of the proximal end portion of the distal end cover 25. A channel 20 having a circular cross section for passing the probe unit 3 is formed inside the hold member 26.

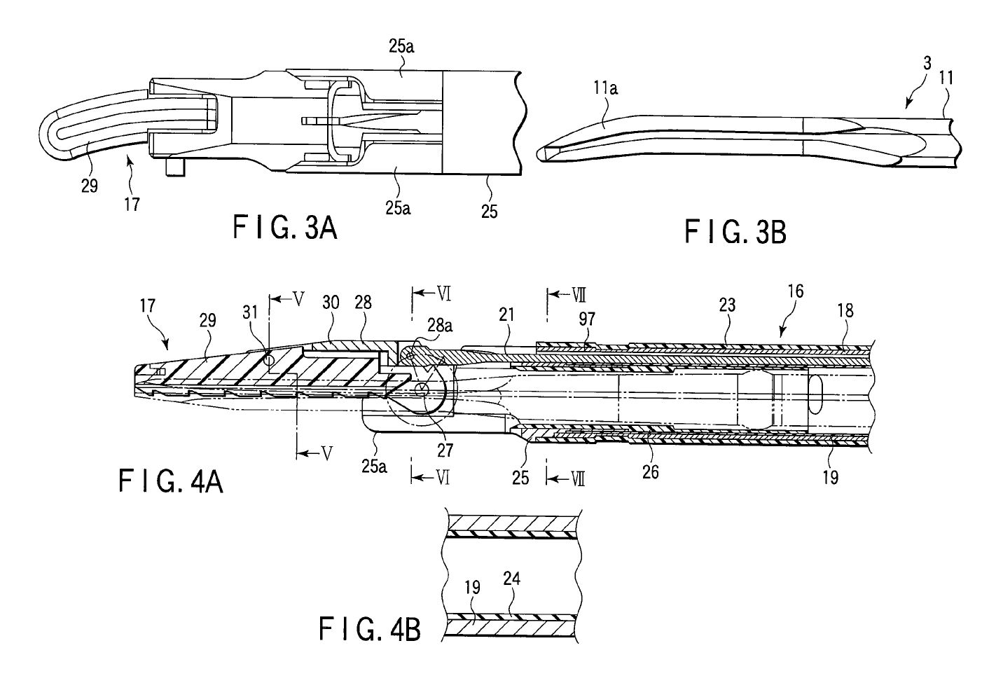

As shown in FIG. 3A, a pair of right and left jaw support portions 25a are formed at a distal end portion of the distal end cover 25 so as to extend toward the front side of the outer cylinder 18. As shown in FIG. 6, a metallic jaw body 28 of the jaw 17 is rotatably attached to the jaw support portions 25a via two support pins 27. The jaw 17, as shown in FIG. 3A, is formed in a substantially J-shaped curved form, which corresponds to the probe distal end portion 11a of the probe unit 3. The jaw 17 is opposed to the probe distal end 11a of the probe unit 3 and supported to be rotatable about the two support pins 27 (see FIG. 6). The jaw 17 is rotated and operated between an open position in which the jaw 17 is rotated in a direction away from the probe distal end 11a of the probe unit 3 and a closed position in which the jaw 17 is rotated in a direction toward the probe distal end 11a of the probe unit 3. By the operation of rotating the jaw 17 to its closed position, a living body tissue is held between the jaw 17 and the probe distal end 11a of the probe unit 3.

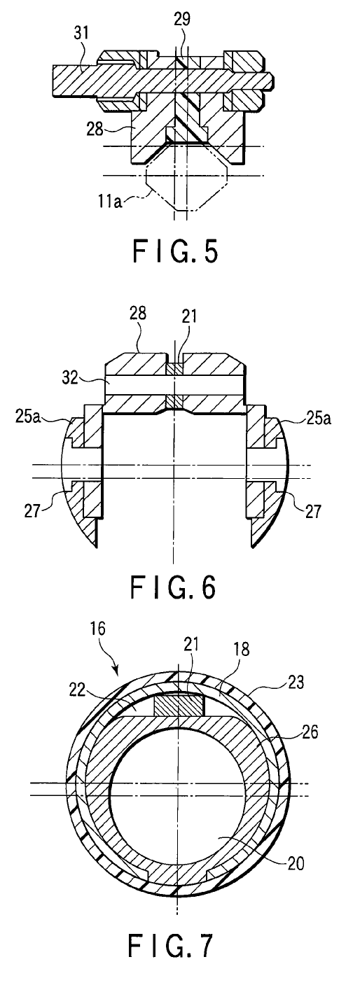

The jaw body 28 includes a hold member 29 which is formed of a resin such as PTFE, and a metallic hold portion attachment member 30 which holds the hold member 29. The hold member 29 is attached to the hold portion attachment member 30 by a pin 31 so as to be rotatable over a predetermined angle (see FIG. 5). Further, as shown in FIG. 4A, a distal end portion of the driving shaft 21 is coupled to the rear end of the jaw body 28 via a pin 28a. The driving shaft 21 extends in the inside of the distal end cover 25, and further extends between the outer cylinder 18 and inner cylinder 19 of the sheath body 16, as shown in FIG. 7, to the proximal end side of the sheath body 16.

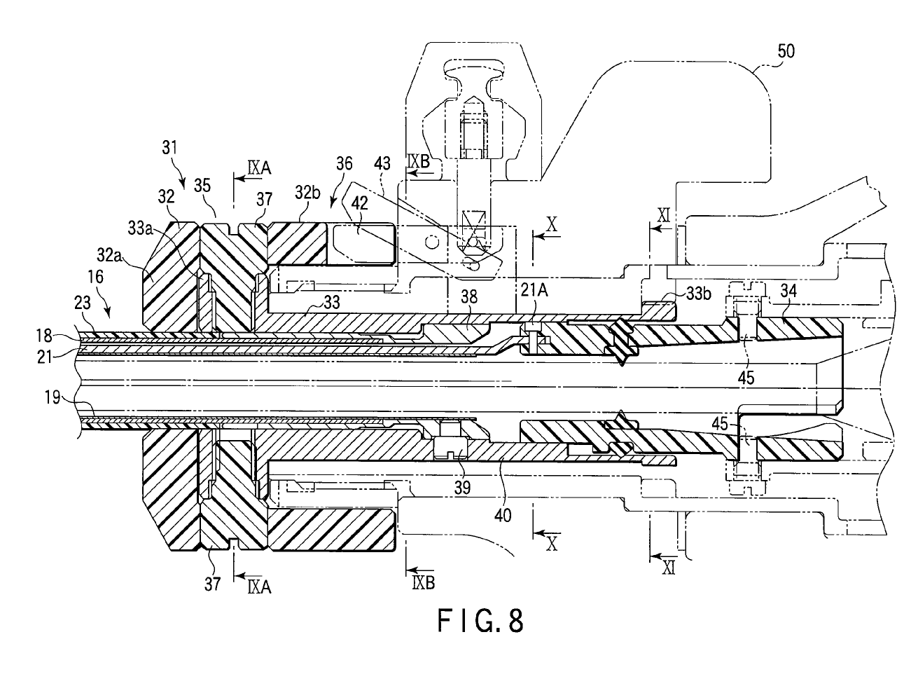

FIG. 8 shows a proximal end portion of the sheath body 16. The proximal end portion of the sheath body 16 is provided with an attachment/detachment mechanism section 31 for attachment/detachment to/from the handle unit 4. The attachment/detachment mechanism section 31 includes a circular cylindrical large-diameter knob member 32 which is formed of a resin material, a guide cylinder body 33 which is formed of a metallic circular cylinder body, and a circular cylindrical connection tube body 34 which is formed of a resin material.

The knob member 32 includes a ring-shaped first fixing portion 32a which is disposed at a front end part, and a circular cylindrical second fixing portion 32b which is disposed at a rear end part. The inner peripheral surface of the first fixing portion 32a is fixed to the outer peripheral surface of the proximal end portion of the sheath body 16. The second fixing portion 32b of the knob member 32 includes a fixing portion 35 of the guide cylinder body 33 that is disposed on the front end side, and an attachment/detachment portion 36 for attachment/detachment to/from the handle unit 4 that is disposed on the rear end part side.

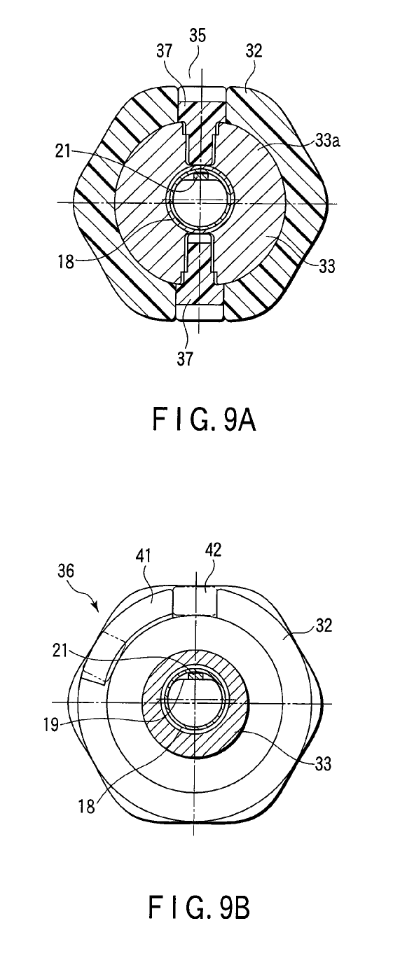

The guide cylinder body 33 includes a large-diameter front-end flange portion 33a which is disposed at the front end part, and an outer peripheral flange portion 33b which is disposed on the rear end part side. As shown in FIG. 9A, the front-end flange portion 33a of the guide cylinder body 33 is fixed to the knob member 32 by two resin-made fixing screws 37 in the state in which the front-end flange portion 33a is inserted in the knob member 32.

A metallic connection pipe 38 is provided inside the guide cylinder body 33. An inner peripheral surface of a front end portion of the connection pipe 38 is fixed by laser welding to the outer cylinder 18 of the sheath body 16. Further, the connection pipe 38 and guide cylinder body 33 are fixed by a metallic fixing screw 39. Thereby, the guide cylinder body 33, fixing screw 39, connection pipe 38, outer cylinder 18, distal end cover 25, support pins 27 and jaw body 28 are electrically connected, and a sheath-unit-side electric path 40 for conduction of a high-frequency electric current is formed.

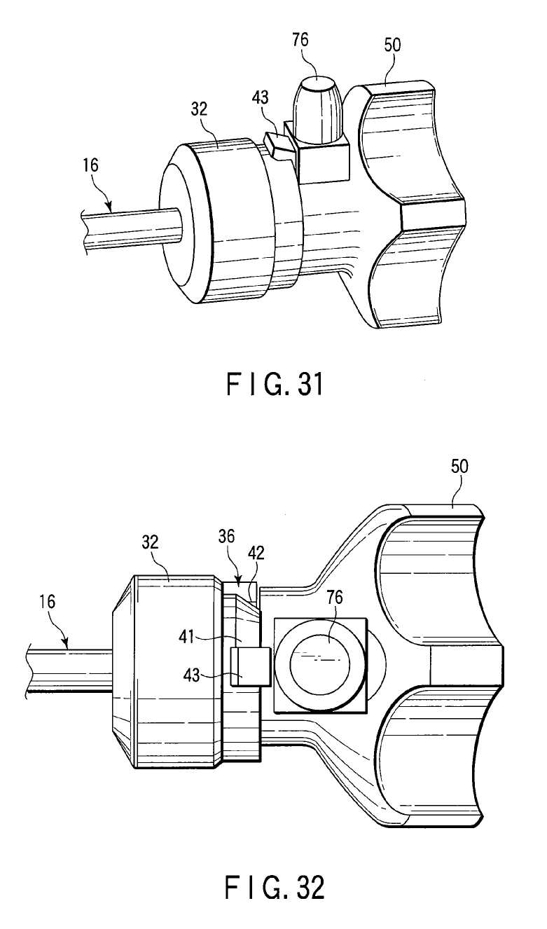

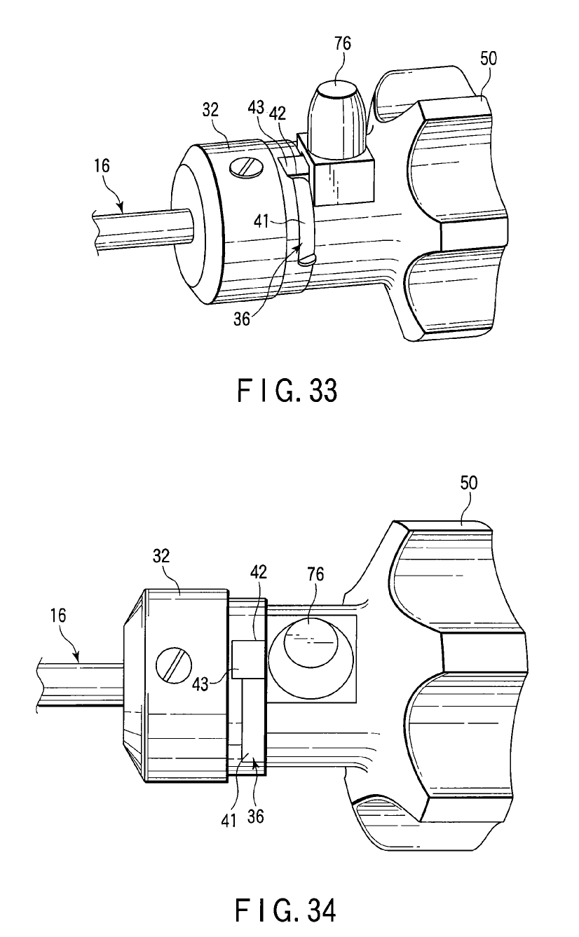

The attachment/detachment portion 36 of the knob member 32 includes an inclined-surface-shaped guide groove 41 which extends in the circumferential direction, as shown in FIG. 9B, and an engaging recess portion 42 which is formed at one end portion of the guide groove 41. The guide groove 41 has a tapered inclined surface having an outside diameter gradually decreasing toward the rear end portion side of the knob member 32. The engaging recess portion 42 is formed of a recess portion having a smaller diameter than the inclined surface of the guide groove 41. An engaging lever 43 (to be described later) on the handle unit 4 side is disengageably engaged in the engaging recess portion 42. FIG. 33 and FIG. 34 show a state in which the engaging lever 43 is engaged in the engaging recess portion 42, and FIG. 31 and FIG. 32 show a disengagement state in which the engaging lever 43 is disengaged from the engaging recess portion 42.

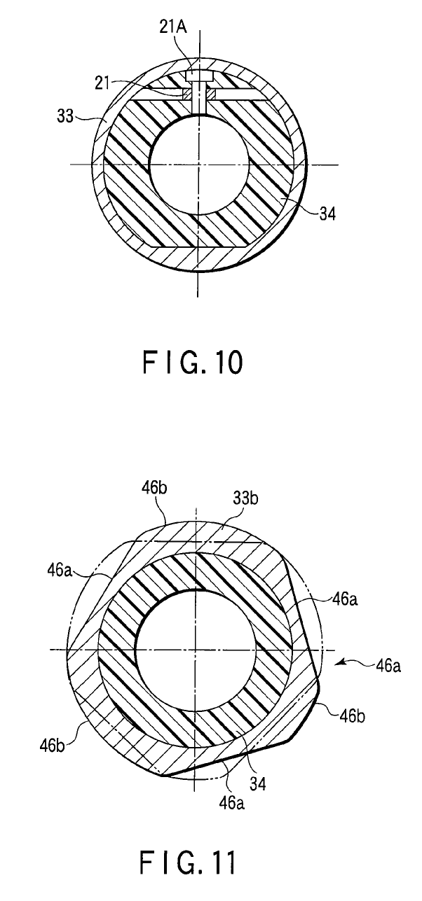

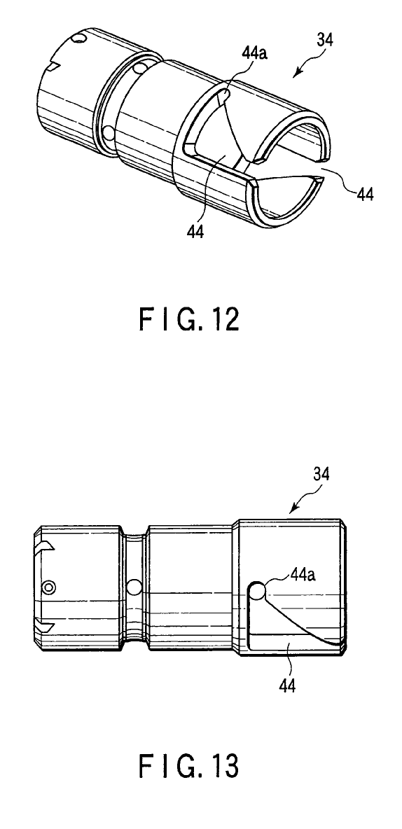

The connection tube body 34 is inserted in the guide cylinder body 33 so as to be slidable in the axial direction of the sheath body 16. A proximal end portion of the driving shaft 21 is fixed to a distal end portion of the connection tube body 34 via a pin 21A (see FIG. 10). A proximal end portion of the connection tube body 34 has two guide grooves 44 as shown in FIGS. 12 and 13. The guide grooves 44 are configured such that engaging pins 45 (to be described later) on the handle unit 4 side are disengageably engaged in the guide grooves 44, respectively. An engaging groove 44a, which restricts movement of the engaging pin 45 in the axial direction of the sheath body 16, is formed at a terminal end portion of the guide groove 44.

The outer peripheral flange portion 33b has a non-circular engaging portion 46. The engaging portion 46 has three cut-out flat-surface portions 46a at a plurality of locations on the circular outer peripheral surface of the outer peripheral flange portion 33b, for example, at three locations in this embodiment. Corner portions 46b, each having a greater diameter than the flat-surface portion 46a, are formed at connection parts between the three flat-surface portions 46a. Thereby, the engaging portion 46 with a substantially triangular cross section is formed on the outer peripheral flange portion 33b. It is not necessary that the non-circular engaging portion 46 have a substantially triangular shape. The non-circular engaging portion 46 may have any other non-circular shape, for instance, a polygon such as a rectangle or a pentagon.

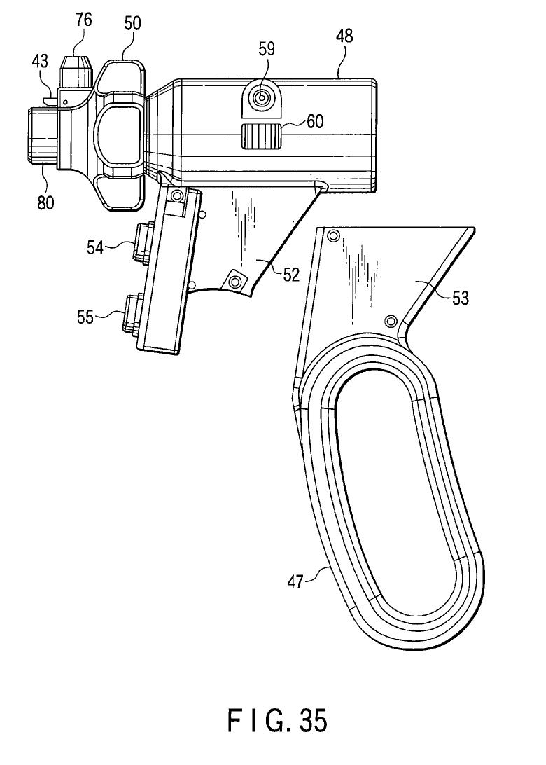

The handle unit 4 mainly includes a stationary handle 47, a hold cylinder 48, a movable handle 49, a rotational operation knob 50 and a handle-unit-side electric path 95 for conduction of a high-frequency electric current. The hold cylinder 48 is provided on the upper part of the stationary handle 47. A switch hold section 51 is provided between the stationary handle 47 and the hold cylinder 48. As shown in FIG. 35, the switch hold section 51 includes a switch attachment section 52 which is fixed to a lower end portion of the hold cylinder 48, and a cover member 53 which is fixed to an upper end portion of the stationary handle 47. The switch attachment section 52 has a plurality of hand switch buttons, for example, two hand switch buttons in this embodiment (e.g. a switch button 54 for incision and a switch button 55 for coagulation), which are push-button switches. A switch 54a for incision, which is operated by the switch button 54 for incision, a switch 55a for coagulation, which is operated by the switch button 55 for coagulation, and a wiring circuit board 92 are assembled in the switch attachment section 52. A wiring line 93a for incision, which has one end connected to the switch 54a for incision, a wiring line 93b for coagulation, which has one end connected to the switch 55a for coagulation, and a ground wiring line 93c, which has one end connected to a common terminal for grounding, are connected to the wiring circuit board 92. These three wiring lines 93a to 93c are looped and assembled in the switch hold section 51.

The movable handle 49 has a substantially U-shaped arm section 56 at an upper part thereof. The U-shaped arm section 56 includes two arms 56a and 56b, as shown in FIG. 20. The movable handle 49 is assembled to the hold cylinder 48 in the state in which the hold cylinder 48 is inserted between the two arms 56a and 56b.

Each of the arms 56a and 56b has a support pin 57 and an operation pin 58. A pin receiving hole portion 59 and a window portion 60 are formed in each of both side portions of the hold cylinder 48. The support pin 57 of each arm 56a, 56b is inserted in the pin receiving hole portion 59 of the hold cylinder 48. Thereby, an upper end portion of the movable handle 49 is rotatably supported on the hold cylinder 48 via the support pins 57.

Finger hook portions 61 and 62 are provided on lower end portions of the stationary handle 47 and movable handle 49, respectively. By hooking the fingers on the finger hook portions 61 and 62 and holding them, the movable handle 49 rotates via the support pins 57 and the movable handle 49 is opened/closed relative to the stationary handle 47.

The operation pins 58 of the movable handle 49 extend into the hold cylinder 48 through the window portions 60 of the hold cylinder 48. An operation force transmission mechanism 63, which transmits an operation force of the movable handle 49 to the driving shaft 21 of the jaw 17, is provided inside the hold cylinder 48.

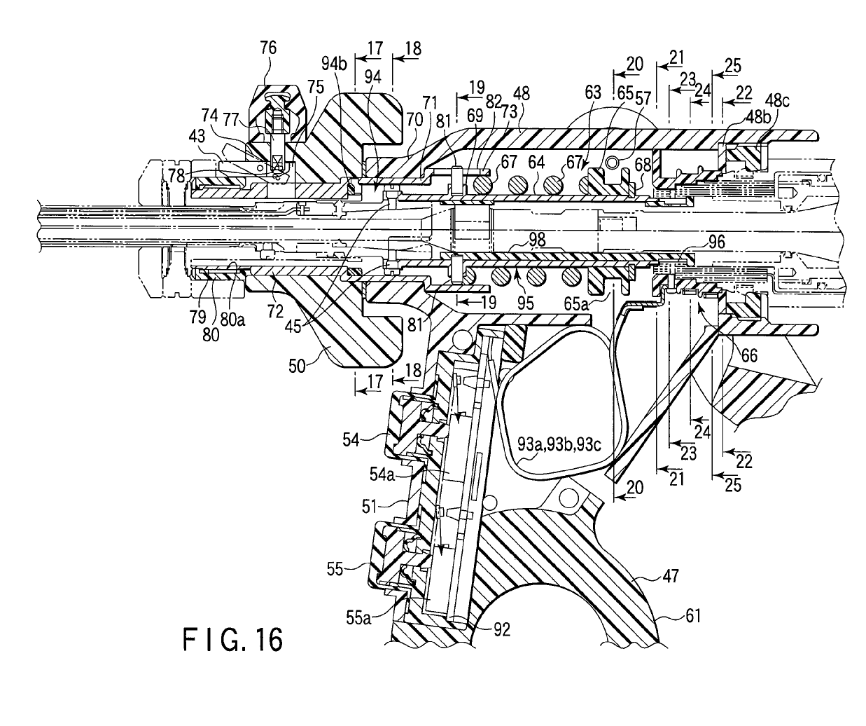

As shown in FIG. 16, the operation force transmission mechanism 63 mainly comprises a metallic circular cylindrical spring receiving member 64 and a resin-made slider member 65. The spring receiving member 64 is disposed coaxially with the center axis of the hold cylinder 48, and extends in the same direction as the direction of insertion of the probe unit 3.

A proximal end portion of the spring receiving member 64 is coupled to a circular cylindrical contact-point unit 66 (to be described later), which is fixed to a proximal end portion of the hold cylinder 48, so as to be rotatably about the axis and to be advancible/retreatable in the same direction as the direction of insertion of the probe unit 3. The above-described pair of engaging pins 45 on the handle unit 4 side are inwardly projectingly provided at a distal end portion of the spring receiving member 64. When the handle unit 4 and sheath unit 5 are coupled, the pair of engaging pins 45 on the handle unit 4 side are disengageably engaged with the engaging grooves 44a at the terminal end portion of the guide grooves 44 of the sheath unit 5.

A coil spring 67, the slider member 65, a stopper 68 and a spring receiver 69 are provided on an outer peripheral surface of the spring receiving member 64. A front end portion of the coil spring 67 is fixed to the spring receiver 69. The stopper 68 restricts the position of movement of a rear end side of the slider member 65. The coil spring 67 is disposed between the spring receiver 69 and the slider member 65 with a fixed amount of mounting force.

An annular engaging groove 65a is formed in a circumferential direction in an outer peripheral surface of the slider member 65. As shown in FIG. 20, the operation pins 58 of the movable handle 49 are inserted and engaged in the engaging groove 65a. If the movable handle 49 is held and the movable handle 49 is closed relative to the stationary handle 47, the operation pins 58 rotate about the support pins 57 in accordance with the rotational operation of the movable handle 49 at this time. The slider member 65, which is in interlock with the rotation of the support pins 57, moves forward in the axial direction. At this time, the spring receiving member 64, which is coupled to the slider member 65 via the coil spring 67, moves forward/backward together with the slider member 65. Thereby, the operation force of the movable handle 49 is transmitted to the connection tube body 34 via the pair of engaging pins 45, and the driving shaft 21 of the jaw 17 moves forward. Thus, the jaw body 20 of the jaw 17 rotates via the support pin 21.

Further, when a living body tissue is clamped between the hold member 29 of the jaw 17 and the probe distal end portion 11a of the probe unit 3 by this operation, the hold member 29 rotates over a certain angle about the pin 31 in accordance with the bending of the probe distal end portion 11a so that force uniformly acts over the entire length of the hold member 29. In this state, ultrasonic is output and a living body tissue, such as a blood vessel, can be coagulated or cut.

An annular bearing portion 70 is formed at a front end portion of the hold cylinder 48. The bearing portion 70 is metallic, and a circular cylindrical rotation transmission member 71 is coupled to the bearing portion 70 so as to be rotatable about the axis. The rotation transmission member 71 includes a projecting portion 72 which projects forward of the bearing portion 70, and a large-diameter portion 73 which extends to the inner side of the hold cylinder 48 from the bearing portion 70.

The rotational operation knob 50 is fitted and fixed on the projecting portion 72. The engaging lever 43 is provided at the front end portion of the rotational operation knob 50. An intermediate portion of the engaging lever 43 is rotatably coupled to the projecting portion 72 via a pin 74. A proximal end portion of the engaging lever 43 extends to the inside of a lever receiving recess portion 75 which is formed in a front surface of the rotational operation knob 50.

An operation button 76 for operating the engaging lever 43 in such a direction as to disengage the engaging lever 43 is provided on an outer peripheral surface of the front end portion of the rotational operation knob 50. An operation pin 77, which is disposed downward, is provided so as to project from the operation button 76. The operation pin 77 extends to the inside of the lever receiving recess portion 75 through a wall hole of the rotational operation knob 50. A proximal end portion of the engaging lever 43 is rotatably coupled to a lower end portion of the operation pin 77 via a pin 78.

A removal prevention ring 80 for the rotational operation knob 50 is provided on a distal end portion of the projecting portion 72. A male threaded portion 79 is formed on the distal end portion of the projecting portion 72. A female threaded portion 80a, which is to be meshed with the male threaded portion 79, is formed on an inner peripheral surface of the removal prevention ring 80. The female threaded portion 80a of the removal prevention ring 80 is meshed and engaged with the male threaded portion 79 of the projecting portion 72, and thereby the rotational operation knob 50 is fixed to the rotation transmission member 71.

As shown in FIG. 19, the spring receiver 69 of the spring receiving member 64 is provided with four metallic positioning pins 81 which project radially outward. An elongated engaging hole portion 82, in which one pin 81 of the spring receiving member 64 is inserted, is formed in the large-diameter portion 73 of the rotation transmission member 71. The engaging hole portion 82 extends in the same direction as the direction of insertion of the probe unit 3. Thereby, when the movable handle 49 is operated, the pin 81 is moved along the engaging hole portion 82 and thus the advancing/retreating movement of the spring receiving member 64 is prevented from being transmitted to the rotation transmission member 71.

On the other hand, when the rotational operation knob 50 is rotated, the rotational movement of the rotation transmission member 71, which rotates together with the rotational operation knob 50, is transmitted to the spring receiving member 64 via the pin 81. Thereby, when the rotational operation knob 50 is rotated, the assembly unit of the rotation transmission member 71, pin 81, spring receiving member 64, slider member 65 and coil spring 67 within the hold cylinder 48 is rotated together with the rotational operation knob 50 as one body about the axis thereof.

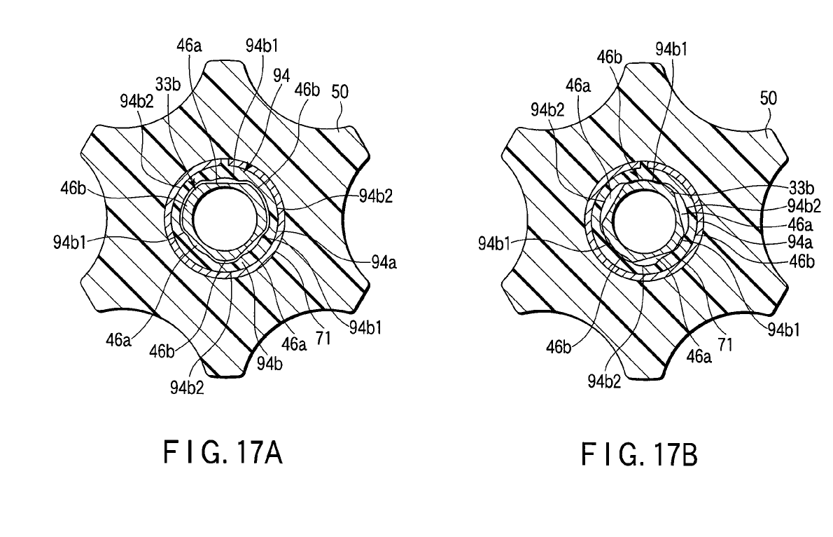

Engaging means 94, which is disengageably engaged with the outer peripheral flange portion 33b of the sheath unit 5, is provided on the inner peripheral surface of the rotation transmission member 71 at a substantially central position in the axial direction. As shown in FIGS. 17A and 17B, the engaging means 94 includes an insertion hole portion 94a in which the outer peripheral flange portion 33b is inserted when the sheath unit 5 and handle unit 4 are coupled, and an electrically conductive rubber ring (urging means) 94b which is disposed within the insertion hole portion 94a.

The shape of the inner peripheral surface of the electrically conductive rubber ring 94b is substantially the same as the shape of the engaging portion 46 of the outer peripheral flange portion 33b. Specifically, the inner peripheral surface of the electrically conductive rubber ring 94b has three cut-out flat-surface portions 94b1 at a plurality of locations on the circular inner peripheral surface, for example, at three locations in this embodiment, and three corner portions 94b2 which are located at connection parts between the three flat-surface portions 94b1 and have greater diameters than the flat-surface portions 94b1. Thereby, the electrically conductive rubber ring 94b has a substantially triangular cross-sectional shape. Thus, as shown in FIG. 17A, the electrically conductive rubber ring 94b is held in a natural, non-compressed position in the positional state in which the inner peripheral surface shape of the electrically conductive rubber ring 94b corresponds to the engaging portion 46 of the outer peripheral flange portion 33b, that is, in the state in which the three corner portions 46b of the outer peripheral flange portion 33b correspond in position to the three corner portions 94b2 of the electrically conductive rubber ring 94b. On the other hand, by rotating the handle unit 4 and the sheath unit 5 relative to each other about the center axis of the sheath unit 5, the position of the electrically conductive rubber ring 94b is switched to a pressure contact position, as shown in FIG. 17B, where the electrically conductive rubber ring 94b is pressed on the three corner portions 46b of the outer peripheral flange portion 33b. At this time, the three corner portions 46b of the outer peripheral flange portion 33b are put in contact with, and pressed by, the three flat-surface portions 94b1 of the electrically conductive rubber ring 94b.

In the present embodiment, at the time of coupling the sheath unit 5 and handle unit 4, when the outer peripheral flange portion 33b of the sheath unit 5 is inserted straight into the electrically conductive rubber ring 94b (see FIG. 31 and FIG. 32), the electrically rubber ring 94b is held in the natural, non-compressed position, as shown in FIG. 17A. At this time, the engaging lever 43 on the handle unit 4 side is held in the state in which the engaging lever 43 rests on the inclined surface of the guide groove 41 of the handle member 32 of the sheath unit 5. Subsequently, the handle member 32 of the sheath unit 5 is rotated about the axis, relative to the handle unit 4. Thereby, as shown in FIG. 33 and FIG. 34, the engaging lever 43 on the handle unit 4 side is inserted and engaged in the engaging recess portion 42 at one end portion of the guide groove 41. At this time, as shown in FIG. 17B, the electrically conductive rubber ring 94b is switched to the pressure contact position where the electrically conductive rubber ring 94b is put in pressure contact with the three corner portions 46b of the outer peripheral flange portion 33b. Thereby, a sheath-unit-side electric path 40 (formed between the guide cylindrical body 33, fixing screw 39, coupling pipe 38, outer cylinder 18, distal end cover 25, support pin 27 and jaw body 28) and a handle-unit-side electric path 95 (formed between the electric contact-point member 96, spring receiving member 64, positioning pin 81 and rotation transmission member 71) are electrically connected via the electrically conductive rubber ring 94b. In this case, a second high-frequency electric path 97, which transmits a high-frequency current, is formed in the coupled body of the sheath unit 5 and handle unit 4.

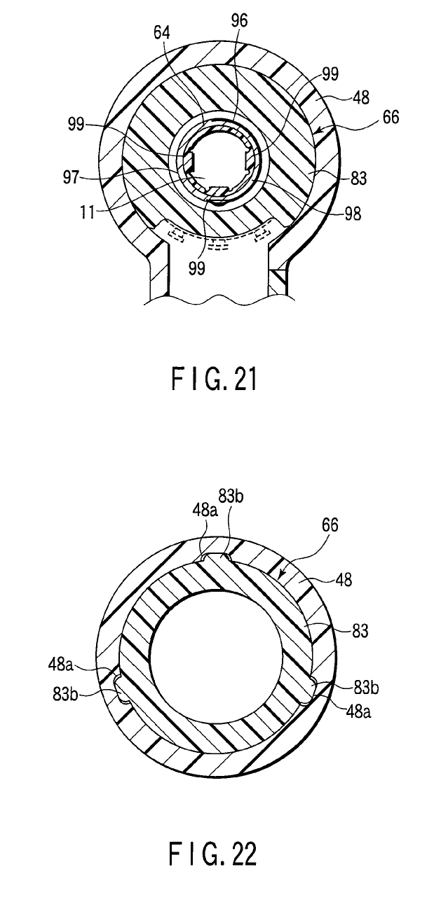

As shown in FIG. 21, the handle unit 4 includes a tubular member 98 which is formed of an insulating material on the inner peripheral surface of the spring receiving member 64. The tubular member 98 is fixed on the inner peripheral surface of the spring receiving member 64. Thereby, when the probe unit 3 and the handle unit 4 are connected, the first high-frequency electric path 13 and the second high-frequency electric path 97 are insulated by the tubular member 98.

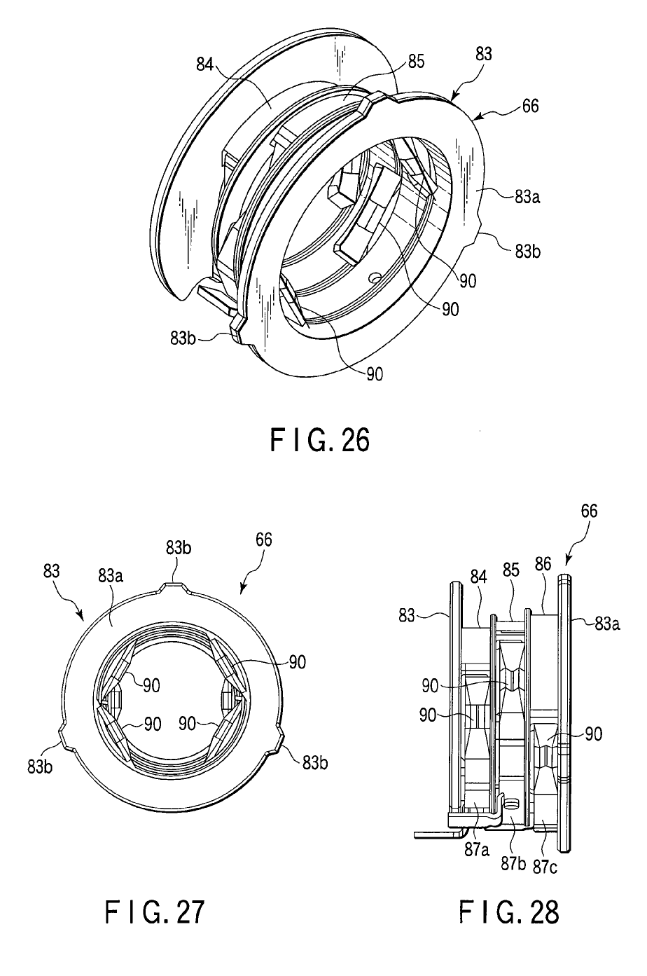

FIGS. 26 to 28 show the circular cylindrical contact-point unit 66. A front end portion of the transducer unit 2 is detachably coupled to the contact-point unit 66. The contact-point unit 66 includes a circular cylindrical electrode hold member 83 which is formed of a resin. As shown in FIG. 28, the electrode hold member 83 includes three (first to third) electrode receiving sections 84, 85 and 86 with different outside diameters. The first electrode receiving section 84 on the distal end side has a smallest diameter, and the third electrode receiving section 86 on the rear end side has a greatest diameter.

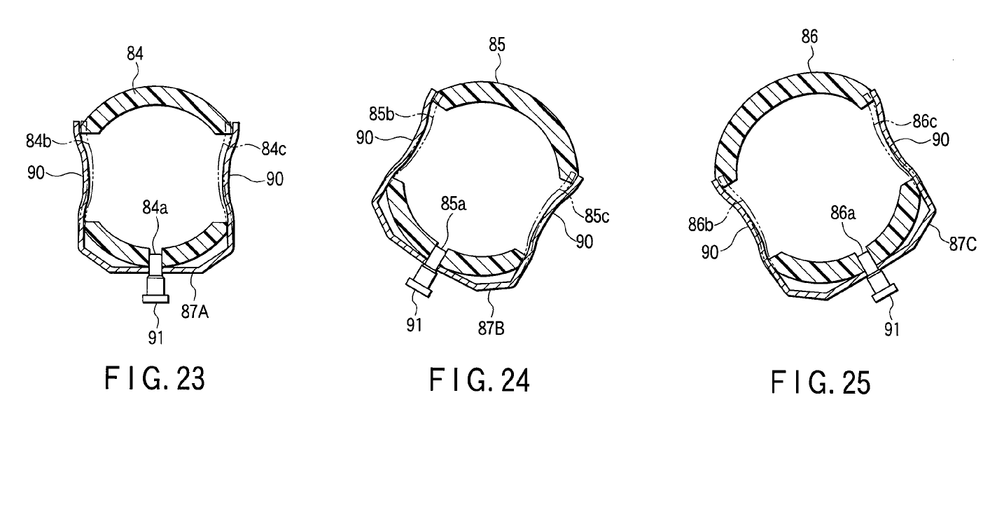

As shown in FIG. 23, the first electrode receiving section 84 has one contact-point member fixing hole 84a, and two through-holes 84b and 84c. A center line of the two through-holes 84b and 84c is set to be perpendicular to a center line of the contact-point member fixing hole 84a.

Similarly, as shown in FIG. 24, the second electrode receiving section 85 has one contact-point member fixing hole 85a, and two through-holes 85b and 85c. As shown in FIG. 25, the third electrode receiving section 86 has one contact-point member fixing hole 86a, and two through-holes 86b and 86c.

The positions of the contact-point member fixing hole 84a of the first electrode receiving section 84, the contact-point member fixing hole 85a of the second electrode receiving section 85 and the contact-point member fixing hole 86a of the third electrode receiving section 86 are displaced in the circumferential direction of the electrode hold member 83.

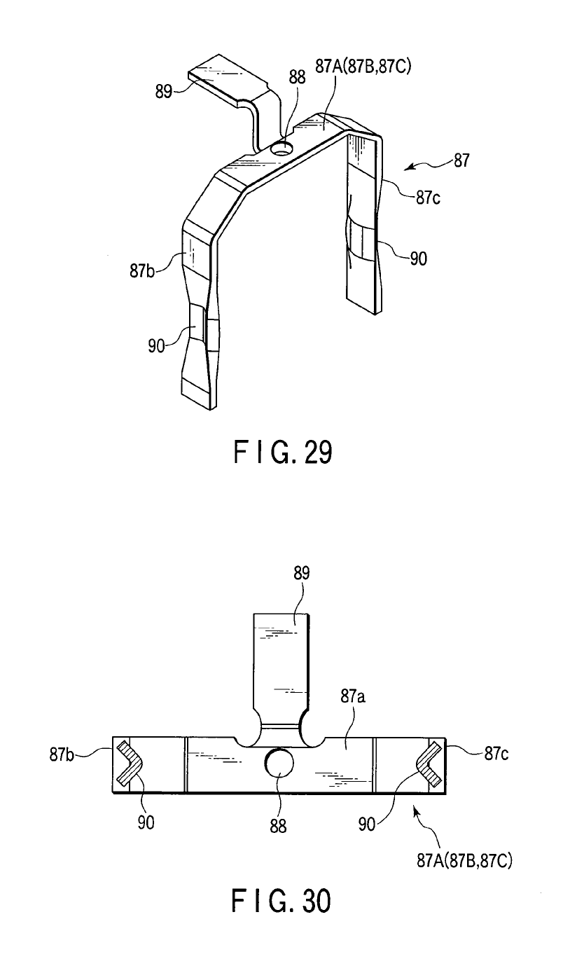

FIG. 29 and FIG. 30 show electrode members 87A, 87B and 87C which are assembled to the first to third electrode receiving sections 84, 85 and 86. These electrode members 87A, 87B and 87C are formed in the same shape. In the description below, only the electrode member 87A, which is assembled to the first electrode receiving section 84, is described. The common parts of the electrode members 87B and 87C of the other second and third electrode receiving sections 85 and 86 are denoted by like reference numerals, and a description thereof is omitted.

The electrode member 87A includes one straight stationary portion 87a and two bend portions 87b and 87C. One bend portion 87b is disposed at one end of the straight stationary portion 87a, and the other bend portion 87c is disposed at the other end of the straight stationary portion 87a. Thereby, as shown in FIG. 29, the electrode member 87A is formed and bent in a substantially U shape.

A hole 88 and an L-shaped wiring connection portion 89 are provided at a central position of the stationary portion 87a. Inwardly curved waist portions 90 are formed at central positions of the two bend portions 87b and 87c.

When the electrode member 87A is assembled to the first electrode receiving section 84, a fixing pin 91 is inserted in the hole 88 of the stationary portion 87a of the electrode member 87A and in the contact-point member fixing hole 85a of the first electrode receiving section 84. The electrode member 87A is fixed to the first electrode receiving section 84 by the fixing pin 91. At this time, the waist portion 90 of one bend portion 87b of the electrode member 87A is disposed in one through-hole 85b of the first electrode receiving section 84, and the waist portion 90 of the other bend portion 87c of the electrode member 87A is disposed in the other through-hole 85c. The same applies when the electrode member 87B is assembled to the second electrode receiving section 85 and the electrode member 87C is assembled to the third electrode receiving section 86.

As shown in FIG. 22, a large-diameter fixing flange portion 83a is formed at a rear end portion of the electrode hold member 83 of the contact-point unit 66. Engaging projection portions 83b are projectingly provided on the outer peripheral surface of the fixing flange portion 83a at a plurality of locations, for example, at three locations in this embodiment. Engaging recess portions 48a are formed in an inner peripheral surface of the rear end portion of the hold cylinder 48 at positions corresponding to the three engaging projection portions 83b of the fixing flange portion 83a. In the case where the electrode hold member 83 is assembled in the hold cylinder 48, the three engaging projection portions 83b of the fixing flange portion 83a are inserted, engaged and fixed in the engaging recess portions 48a of the hold cylinder 48. Thereby, the rotation of the electrode hold member 83 about the axis thereof, relative to the hold cylinder 48, is restricted.

A stepped portion 43b, which comes in contact with the fixing flange portion 83a of the electrode hold member 83, is formed on the hold cylinder 48. The electrode hold member 83 is fixed to the hold cylinder 48 by a fixing screw 48c in the state in which the fixing flange portion 83a of the electrode hold member 83 abuts upon the stepped portion 43b. Thereby, the axial movement of the electrode hold member 83, relative to the hold cylinder 48, is restricted.

End portions of three wiring lines 93a to 93c, which are assembled in the switch hold section 51, are connected to the wiring connection portions 89 of the three electrode members 87A, 87B and 87C that are assembled to the contact-point unit 66.

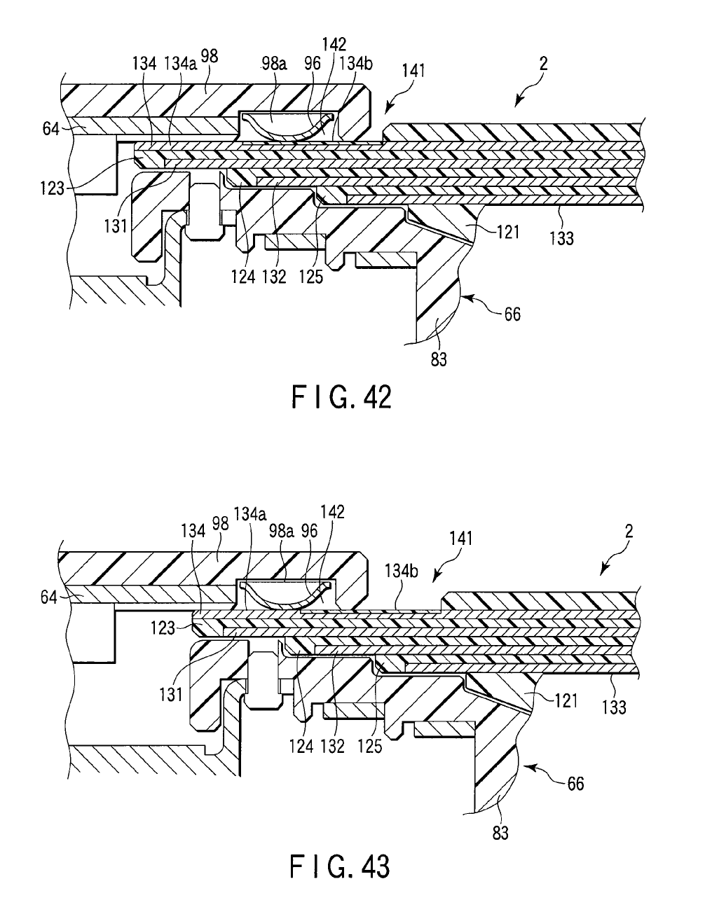

As shown in FIG. 42, a recess-shaped electric contact-point attachment groove 98a is formed in an outer peripheral surface of a rear end portion of the tubular member 98. As shown in FIG. 21, a substantially C-shaped electric contact-point member 96, which is formed of a metallic plate spring, is attached to the electric contact-point attachment groove 98a. The electric contact-point member 96 is connected to the outer-peripheral surface of the proximal end portion of the spring receiving member 64.

An inner peripheral surface of the tubular member 98 has three engaging projection portions 99 which correspond to the three engaging recess portions 15 (see FIG. 37) of the flange portion 14 of the probe unit 3. When the probe unit 3 and handle unit 4 are connected, the three engaging projection portions 99 of the tubular member 98 are disengageably engaged with the three engaging recess portions 15 of the flange portion 14 of the probe unit 3. Thereby, the rotational-directional position between the probe unit 3 and the tubular member 98 of the handle unit 4 is restricted. Hence, when the rotational operation knob 50 is rotated, the coupled body of the probe unit 3 and transducer unit 2 is rotated as one body together with the assembly unit within the hold cylinder 48.

The engaging section between the flange portion 14 of the probe unit 3 and the tubular member 98 is not limited to the above-described structure. For example, the tubular member 98 may be formed to have a D-shaped cross section, and the flange portion 14 of the probe unit 3 may be formed to have a corresponding D-shaped cross section.

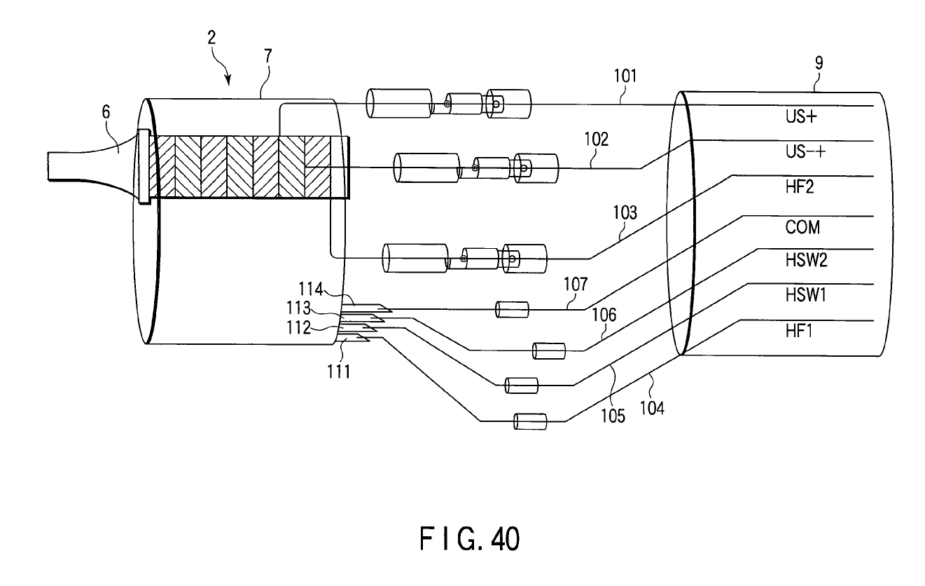

FIG. 40 shows the connection of internal wiring between the single cable 9 at the rear end of the transducer unit 2 and the transducer unit 2. As shown in FIG. 40, two wiring lines 101 and 102 for the ultrasonic transducer, two wiring lines 103 and 104 for high-frequency power and three wiring lines 105, 106 and 107, which are connected to a wiring circuit board 92 within the switch hold section 51, are assembled in the single cable 9 at the rear end of the transducer unit 2. Distal end portions of the two wiring lines 101 and 102 for the ultrasonic transducer are connected to the ultrasonic transducer 6. A distal end portion of one wiring line 103 for high-frequency power is connected to the ultrasonic transducer 6.

First to fourth electrically conductive plates 111 to 114 for electric connection are provided at the rear end of the transducer unit 2. A distal end portion of the other wiring line 104 for high-frequency power is connected to the first conductive plate 111. The three wiring lines 105, 106 and 107 are connected to the second to fourth conductive plates 112 to 114.

FIG. 41 shows the internal structure of a front end portion of the transducer unit 2. A connection cylindrical portion 121 is formed at the distal end portion of the transducer cover 7. A C-ring 122 having a partly cut-out annular plate shape is mounted on the outer peripheral surface of the connection cylindrical body 121. Three (first to third) cylindrical portions 123 to 125 with different outside diameters are projectingly provided on the inside of the connection cylindrical portion 121. The first cylindrical portion 123 has a smallest outside diameter and has a greatest length of projection from the distal end of the connection cylindrical body 121. The second cylindrical portion 124 has an outside diameter, which is greater than the outside diameter of the first cylindrical portion 123, and has a length of projection from the distal end of the connection cylindrical body 121, which is less than the length of projection of the first cylindrical portion 123. The third cylindrical portion 125 has a greatest outside diameter and has a length of projection from the distal end of the connection cylindrical body 121, which is less than the length of projection of the second cylindrical portion 124.

A first cylindrical contact-point member 131 is mounted on the outer peripheral surface of the first cylindrical portion 123. Similarly, a second cylindrical contact-point member 132 is mounted on the outer peripheral surface of the second cylindrical portion 124, and a third cylindrical contact-point member 133 is mounted on the outer peripheral surface of the third cylindrical portion 125. The second conductive plate 112 is connected to the first contact-point member 131, the third conductive plate 113 is connected to the second contact-point member 132, and the fourth conductive plate 114 is connected to the third contact-point member 133.

A cylindrical fourth contact-point member 134 is mounted on the inner peripheral surface of the first cylindrical body 123. The fourth contact-point member 134 is connected to the first conductive plate 111.

When the handle unit 4 and the transducer unit 2 are coupled, the contact-point unit 66 of the handle unit 4 and the front end portion of the transducer unit 2 are connected. At this time, the electrode member 87A of the contact-point unit 66 and the first contact-point member 131 of the transducer unit 2 are connected. At the same time, the electrode member 87B of the contact-point unit 66 and the second contact-point member 132 of the transducer unit 2 are connected, the electrode member 87C of the contact-point unit 66 and the third contact-point member 133 of the transducer unit 2 are connected, and the C-shaped electric contact-point member 96 of the tubular member 98 and the fourth contact-point member 134 of the transducer unit 2 are connected.

Furthermore, the handpiece 1 of the present embodiment includes an output mode switching section (output mode switching means) 141 (see FIG. 42, 43). The output mode switching section 141 is switch means for selectively switching the output of the surgical operating apparatus between a bipolar mode in which the probe distal end 11a and jaw 17 are used as high-frequency electrodes and driven in a bipolar mode, and a probe-only output mode in which energy is output from only the probe distal end 11a.

The output mode switching section 141 is provided by making use of some of the movable parts, i.e. the slider member 65, coil spring 67, spring receiving member 64, tubular member 98 and connection tube body 34, of the operation force transmission member which is advanced/retreated in the axial direction of the probe 11 in accordance with the operation of the movable handle 49 and transmits the operational force of the movable handle 49 to the jaw 17. Specifically, in the present embodiment, the output mode switching section 141 having the structure, which will be described below, is provided at the connection part between the C-shaped electric contact-point member 96 of the tubular member 98 of the handle unit 4 and the fourth contact-point member 134 of the transducer unit 2.

As is shown in FIG. 42, the surface (inner peripheral surface) of the fourth contact-point member 134 is provided with an electrically conductive portion 134a on which metallic material is exposed, and an insulative portion 134b which is configured such that an insulating coating is applied to a surface of metallic material. The insulative portion 134b is formed of, e.g. a fluorine-based coating or a DLC coating. The conductive portion 134a is disposed on the front side of the fourth contact-point member 134, and the insulative portion 134b is disposed in rear of the conductive portion 134a. When the movable handle 49 is operated, the tubular member 98 moves in the axial direction of the probe 11. In accordance with the operation of the tubular member 98, a contact part (one of the conductive portion 134a and insulative portion 134b of the fourth contact-point member 134) of the fourth contact-point member 134, which comes in contact with the C-shaped electric contact-point member 96, is switched. At this time, switching is effected between the state in which the conductive portion 134a of the fourth contact-point member 134 is in contact with the C-shaped electric contact-point member 96, and the state in which the insulative portion 134b of the fourth contact-point member 134 is in contact with the C-shaped electric contact-point member 96. Thereby, a contact-point switching section 142 is formed, which opens/closes the contact point that switches on/off the electrical conduction state of a high-frequency current to the jaw 17.

FIG. 42 shows the shift state of the output mode switching section 141 in the case where the jaw 17 is opened. In this state, the movable handle 49 is rotated in a direction away from the stationary handle 47, and the tubular member 98 is moved rearward. At this time, the C-shaped electric contact-point member 96 of the tubular member 98 is in contact with the insulative portion 134b of the fourth contact-point member 134 of the transducer unit 2. Accordingly, electrical connection is cut off between the C-shaped electric contact-point member 96 of the tubular member 98 and the fourth contact-point member 134 of the transducer unit 2. In this case, the supply of high-frequency current to the jaw 17 is turned off. Thus, in this state, the output mode switching section 141 is switched to the probe-only output mode. In the probe-only output mode, only ultrasonic from the transducer 6 is output to the probe 11.

FIG. 43 shows the shift state of the output mode switching section 141 in the case where the jaw 17 is closed. In this state, the movable handle 49 is rotated in a direction toward the stationary handle 47, and the tubular member 98 is moved forward. At this time, the C-shaped electric contact-point member 96 of the tubular member 98 is in contact with the conductive portion 134a of the fourth contact-point member 134 of the transducer unit 2. Accordingly, electrical connection is established in a current conductive state between the C-shaped electric contact-point member 96 of the tubular member 98 and the fourth contact-point member 134 of the transducer unit 2. In this state, the output mode switching section 141 is switched to the bipolar mode. In the bipolar mode, a high-frequency current is supplied to the jaw 17. In this state, the handpiece 1 of the ultrasonic therapeutic apparatus is driven in the bipolar mode in which the probe 11 and jaw 17 are used as high-frequency electrodes. In this case, such a structure may be adopted that ultrasonic is output as energy from the probe 11 at the same time.

Next, the operation of the present embodiment is described. The handpiece 1 of the ultrasonic therapeutic apparatus of the present embodiment, as shown in FIG. 2, comprises four units, namely, the transducer unit 2, probe unit 3, handle unit 4 and sheath unit 5, which are detachable. When the handpiece 1 is used, the transducer unit 2 and the probe unit 3 are coupled. Thereby, the first high-frequency electric path 13, which transmits a high-frequency current to the coupled body of the transducer unit 2 and probe unit 3, is formed.

Subsequently, the handle unit 4 and the sheath unit 5 are coupled. When the handle unit 4 and sheath unit 5 are coupled, the connection tube body 34 is inserted in the rotation transmission member 71 of the handle unit 4 in the state in which the handle member 32 of the sheath unit 5 is held. When the sheath unit 5 and handle unit 4 are coupled, the engaging lever 43 on the handle unit 4 side is held in the state in which the engaging lever 43 rests on the inclined surface of the guide groove 41 of the handle member 32 of the sheath unit 5, as shown in FIG. 31 and FIG. 32. At this time, as shown in FIG. 17A, the electrically conductive rubber ring 94b is held in the positional state in which the inner peripheral surface shape of the electrically conductive rubber ring 94b corresponds to the engaging portion 46 of the outer peripheral flange portion 33b, that is, in the state in which the three corner portions 46b of the outer peripheral flange portion 33b correspond in position to the three corner portions 94b2 of the electrically conductive rubber ring 94b. Accordingly, the outer peripheral flange portion 33b of the sheath unit 5 is inserted straight into the electrically conductive rubber ring 94b. At the time of this insertion operation, as shown in FIG. 17A, the conductive rubber ring 94b is held in the natural, non-compressed position. In this state, the sheath-unit-side electric path 40 and the handle-unit-side electric path 95 are not electrically connected.

Subsequently, following this insertion operation, the handle member 32 of the sheath unit 5 is rotated about the axis thereof, relative to the handle unit 4. By this operation, as shown in FIG. 33 and FIG. 34, the engaging lever 43 on the handle unit 4 side is inserted and engaged in the engaging recess portion 42 at one end portion of the guide groove 41. At this time, as shown in FIG. 17B, the electrically conductive rubber ring 94b is switched to the pressure contact position where the electrically conductive rubber ring 94b is put in pressure contact with the three corner portions 46b of the outer peripheral flange portion 33b. Thereby, the sheath-unit-side electric path 40 and the handle-unit-side electric path 95 are electrically connected via the electrically conductive rubber ring 94b. As a result, the second high-frequency electric path 97, which transmits a high-frequency current, is formed in the coupled body of the sheath unit 5 and handle unit 4.

When the sheath unit 5 is rotated about the axis thereof, the pair of engaging pins 45 on the handle unit 4 side are, at the same time, disengageably engaged in the engaging groove 44a at the terminal end portion of the guide groove 44 of the sheath unit 5. Thereby, the spring receiving member 64 on the handle unit 4 side and the connection tube body 34 on the sheath unit 5 side are coupled via the engaging pins 45. As a result, the operation force on the handle unit 4 side at the time when the movable handle 49 is closed relative to the stationary handle 47 can be transmitted to the driving shaft 21 of the jaw 17 on the sheath unit 5 side. This state is the coupled state between the sheath unit 5 and the handle unit 4.

Thereafter, the coupled body of the sheath unit 5 and handle unit 4 and the coupled body of the ultrasonic transducer 6 and probe unit 3 are assembled as one body. In this assembling work, the contact-point unit 66 of the handle unit 4 is connected to the front end portion of the transducer unit 2. At this time, the electrode member 87A of the contact-point unit 66 and the first contact-point member 131 of the transducer unit 2 are connected. At the same time, the electrode member 87B of the contact-point unit 66 and the second contact-point member 132 of the transducer unit 2 are connected, the electrode member 87C of the contact-point unit 66 and the third contact-point member 133 of the transducer unit 2 are connected, and the C-shaped electric contact-point member 96 of the tubular member 98 and the fourth contact-point member 134 of the transducer unit 2 are connected. Thereby, the second high-frequency electric path 97 of the coupled body of the sheath unit 5 and handle unit 4 is connected to the wiring line 104 for high-frequency power within the cable 9. Further, the three wiring lines 105, 106 and 107 within the cable 9 are connected to the wiring circuit board 92 within the switch hold section 51. This state is the completion state of the assembly of the handpiece 1.

When the handpiece 1 is used, the movable handle 49 is opened/closed relative to the stationary handle 47, and the driving shaft 21 is axially moved in interlock with the operation of the movable handle 49. The jaw 17 is opened/closed, relative to the probe distal end 11a of the probe unit 3, in interlock with the advancing/retreating movement of the driving shaft 21 in its axial direction.

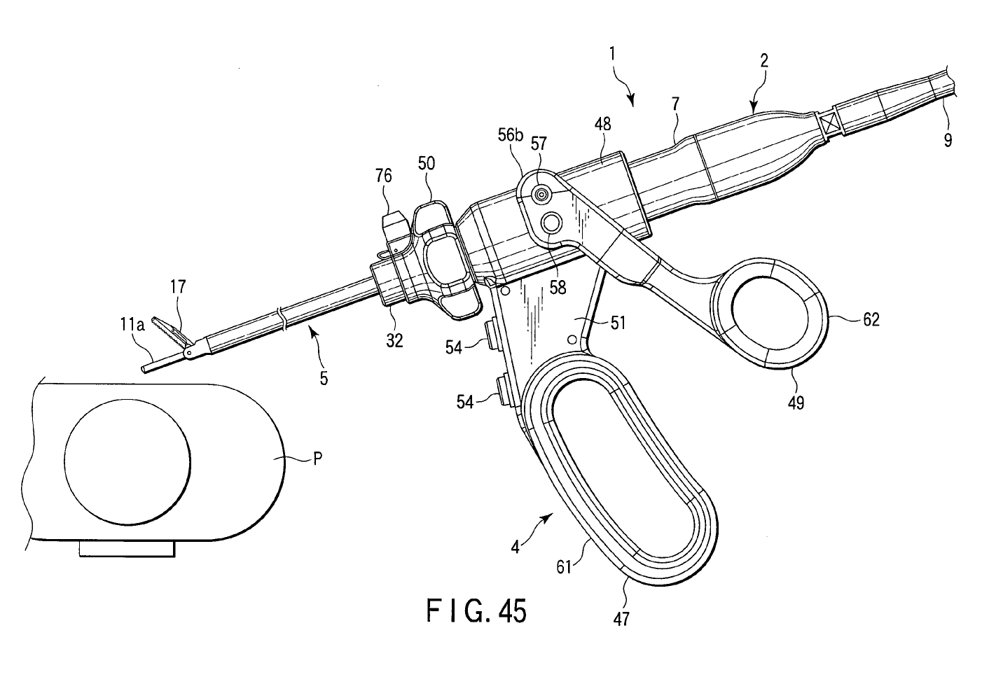

In the case where the movable handle 49 is rotated in a direction away from the stationary handle 47 (“opening operation time”), the driving shaft 21 is pulled rearward in interlock with the operation of the movable handle 4. Accordingly, the jaw 17 is opened. At this time, the output mode switching section 141 is switched to the probe-only output mode shown in FIG. 42. In this case, since the C-shaped electric contact-point member 96 of the tubular member 98 is in contact with the insulative portion 134b of the fourth contact-point member 134 of the transducer unit 2, electrical connection is cut off between the C-shaped electric contact-point member 96 of the tubular member 98 and the fourth contact-point member 134 of the transducer unit 2. As a result, since the supply of high-frequency current to the jaw 17 is turned off, only ultrasonic from the transducer 6 is output to the probe 11. Thus, in this case, as shown in FIG. 45, therapeutic treatment is conducted on a patient P with only ultrasonic vibration which is output from the probe distal end 11a. In this case, for example, therapeutic treatment, such as puncture, can be performed by using cavitation of the probe 11.

In the case where the movable handle 49 is rotated in a direction toward the stationary handle 47 (“closing operation time”), the driving shaft 21 is pushed forward in interlock with the operation of the movable handle 4, and the jaw 17 is moved in the direction of closing. Thus, a living body tissue can be clamped between the probe distal end 11a and the jaw 17. At this time, the output mode switching section 141 is switched to the bipolar mode shown in FIG. 43. In this case, since the C-shaped electric contact-point member 96 of the tubular member 98 is in contact with the conductive portion 134a of the fourth contact-point member 134 of the transducer unit 2, electrical connection is established in an electrically conductive state between the C-shaped electric contact-point member 96 of the tubular member 98 and the fourth contact-point member 134 of the transducer unit 2. As a result, a high-frequency current is supplied to the jaw 17. In this state, the handpiece 1 of the ultrasonic therapeutic apparatus is driven in the bipolar mode in which the probe 11 and jaw 17 are used as high-frequency electrodes. At this time, by outputting ultrasonic as energy from the probe 11 at the same time, strong coagulation or quick incision can be performed on the living body tissue between the probe distal end 11a and the jaw 17.

With the above-described structure, the following advantageous effects can be obtained. Specifically, the handpiece 1 of the ultrasonic therapeutic apparatus of the present embodiment is provided with the output mode switching section 141. In accordance with the operation of the movable handle 49, the output of the surgical operating apparatus is selectively switched between the bipolar mode in which the probe distal end 11a and jaw 17 are used as high-frequency electrodes and driven in a bipolar mode, and the probe-only output mode in which energy is output from only the probe distal end 11a. In the case where the movable handle 49 is rotated in a direction away from the stationary handle 47 (“opening operation time”), the output mode switching section 141 is switched to the probe-only output mode shown in FIG. 42. In this case, in the present embodiment, since the supply of high-frequency current to the jaw 17 is turned off, only ultrasonic from the transducer 6 is output to the probe 11. Accordingly, in this case, as shown in FIG. 45, therapeutic treatment is conducted on the patient P with only ultrasonic vibration which is output from the probe distal end 11a. Thus, for example, therapeutic treatment, such as puncture, can be performed by using cavitation of the probe 11.

In the case where the movable handle 49 is rotated in a direction toward the stationary handle 47 (“closing operation time”), the output mode switching section 141 is switched to the bipolar mode shown in FIG. 43. In this case, a high-frequency current is supplied to the jaw 17. In this state, the handpiece 1 of the ultrasonic therapeutic apparatus is driven in the bipolar mode in which the probe 11 and jaw 17 are used as high-frequency electrodes. At this time, by outputting ultrasonic as energy from the probe 11 at the same time, strong coagulation or quick incision can be performed on the living body tissue between the probe distal end 11a and the jaw 17.

Therefore, in the handpiece 1 of the ultrasonic therapeutic apparatus of the present embodiment, even with the use of the single handpiece 1, selective switching can easily be effected between the bipolar mode and the probe-only output mode in accordance with the operation of the movable handle 49, for example, depending on the phase in use, and proper therapeutic treatment can be performed. Thus, there is no need to perform such a time-consuming operation that an operation is continued after exchanging a monopolar therapeutic device and a bipolar therapeutic device. As a result, compared to the case of continuing an operation by exchanging the monopolar therapeutic device and bipolar therapeutic device, it is possible to improve the operability for the user and to prevent an increase in total time for therapeutic treatment in a surgical operation.

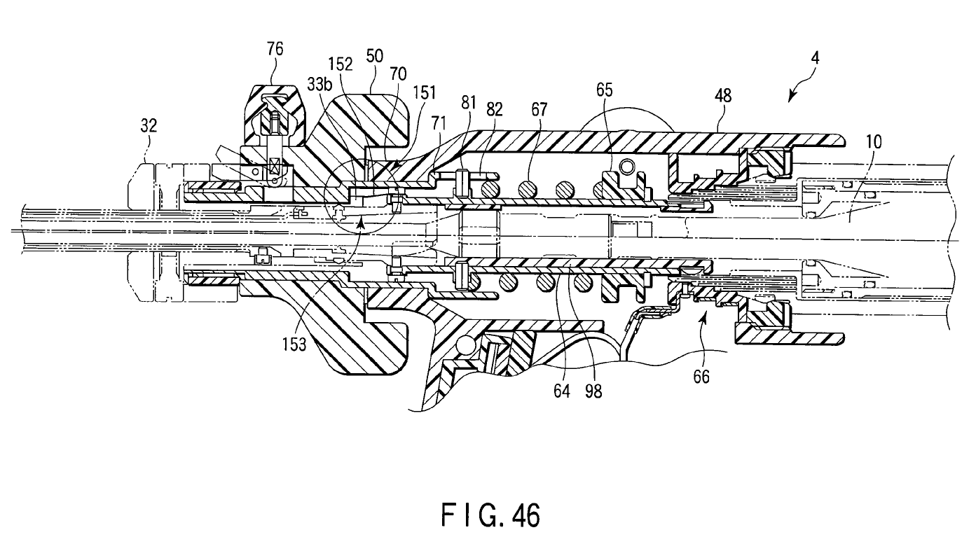

FIG. 46 to FIG. 48 show a second embodiment of the present embodiment. FIG. 46 is a longitudinal cross-sectional view showing an internal structure of a handpiece 1 of an ultrasonic therapeutic apparatus according to the present embodiment. The present embodiment is provided with an output mode switching section 151 which is configured differently from the handpiece 1 of the first embodiment (see FIG. 1 to FIG. 45). In the other structural parts, the second embodiment is substantially the same as the first embodiment (see FIG. 1 to FIG. 45). Thus, in FIG. 46 to FIG. 48, the parts common to those in the first embodiment are denoted by like reference numerals, and a description thereof is omitted.

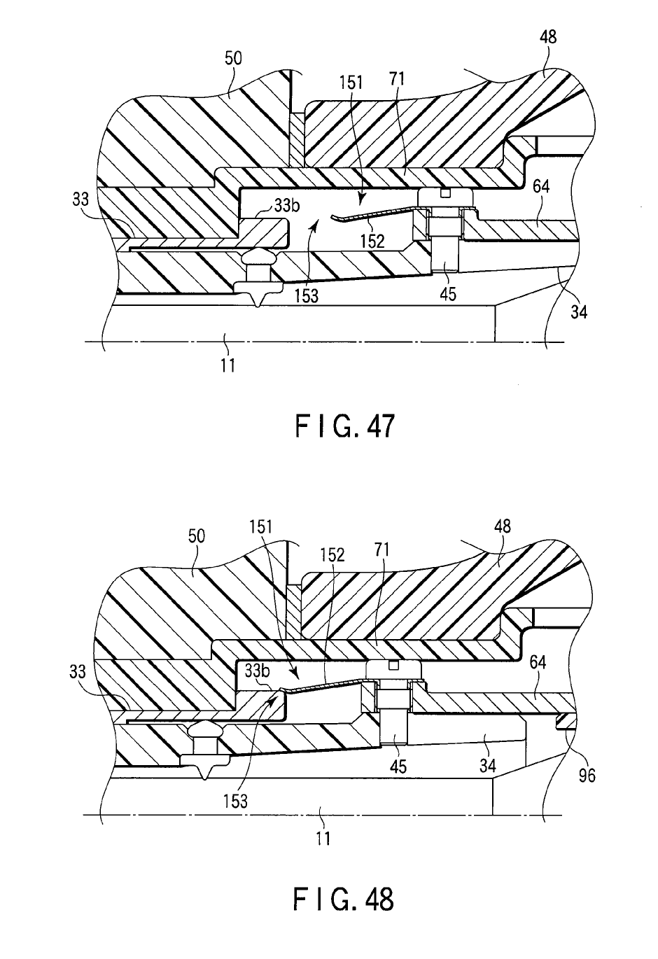

As shown in FIG. 47 and FIG. 48, the output mode switching section 151 of the present embodiment is provided between the spring receiving member 64 on the handle unit 4 side and the outer peripheral flange portion 33b of the guide cylinder body 33 of the sheath unit 5. In the spring receiving member 64, a contact-point member 152 of a metallic plate, which extends forward, is attached to the engaging pin 45 at the distal end portion thereof. The contact-point member 152 is detachably put in contact with the outer peripheral flange portion 33b of the guide cylinder body 33 of the sheath body 16 when the movable handle 49 is operated.

When the movable handle 49 is operated, the spring receiving member 64 moves in the axial direction of the probe 11. In accordance with the operation of the spring receiving member 64, switching is effected between the state in which the contact-point member 152 of the spring receiving member 64 is in contact with the outer peripheral flange portion 33b of the guide cylinder body 33, as shown in FIG. 48, and the state in which the contact-point member 152 of the spring receiving member 64 is not in contact with the outer peripheral flange portion 33b of the guide cylinder body 33, as shown in FIG. 47. The contact-point member 152 constitutes a contact-point switching section 153 which opens/closes the contact point that switches on/off the electrical conduction state of a high-frequency current to the jaw 17 in accordance with the operation of the spring receiving member 64 which moves in accordance with the operation of the movable handle 49. In the present embodiment, the rotation transmission member 71 at the front end portion of the hold cylinder 48 is formed of an insulative resin material.

FIG. 47 shows the shift state of the output mode switching section 151 in the case where the jaw 17 is opened. In this state, the movable handle 49 is rotated in a direction away from the stationary handle 47, and the spring receiving member 64 is moved rearward. At this time, the contact-point member 152 of the spring receiving member 64 is separated from the outer peripheral flange portion 33b of the guide cylinder body 33. Accordingly, electrical connection is cut off between the contact-point member 152 of the spring receiving member 64 and the outer peripheral flange portion 33b of the guide cylinder body 33. In this case, the supply of high-frequency current to the jaw 17 is turned off. Thus, in this state, the output mode switching section 151 is switched to the probe-only output mode. In the probe-only output mode, only ultrasonic from the transducer 6 is output to the probe 11.

FIG. 48 shows the shift state of the output mode switching section 151 in the case where the jaw 17 is closed. In this state, the movable handle 49 is rotated in a direction toward the stationary handle 47, and the spring receiving member 64 is moved forward. At this time, the contact-point member 152 of the spring receiving member 64 is in contact with the outer peripheral flange portion 33b of the guide cylinder body 33. Accordingly, electrical connection is established in an electrically conductive state between the contact-point member 152 of the spring receiving member 64 and the outer peripheral flange portion 33b of the guide cylinder body 33. In this state, the output mode switching section 151 is switched to the bipolar mode. In the bipolar mode, a high-frequency current is supplied to the jaw 17. In this state, the handpiece 1 of the ultrasonic therapeutic apparatus is driven in the bipolar mode in which the probe 11 and jaw 17 are used as high-frequency electrodes. In this case, such a structure may be adopted that ultrasonic is output as energy from the probe 11 at the same time.

With the above-described structure, the following advantageous effects can be obtained. Specifically, the handpiece 1 of the ultrasonic therapeutic apparatus of the present embodiment is provided with the output mode switching section 151. In accordance with the operation of the movable handle 49, the output of the surgical operating apparatus is selectively switched between the bipolar mode in which the probe distal end 11a and jaw 17 are used as high-frequency electrodes and driven in a bipolar mode, and the probe-only output mode in which energy is output from only the probe distal end 11a. In the case where the movable handle 49 is rotated in a direction away from the stationary handle 47 (“opening operation time”), the output mode switching section 151 is switched to the probe-only output mode shown in FIG. 47. In this case, in the present embodiment, since the supply of high-frequency current to the jaw 17 is turned off, only ultrasonic from the transducer 6 is output to the probe 11. Accordingly, in this case, as shown in FIG. 45, therapeutic treatment is conducted on the patient P with only ultrasonic vibration which is output from the probe distal end 11a. Thus, for example, therapeutic treatment, such as puncture, can be performed by using cavitation of the probe 11.

In the case where the movable handle 49 is rotated in a direction toward the stationary handle 47 (“closing operation time”), the output mode switching section 151 is switched to the bipolar mode shown in FIG. 48. In this case, a high-frequency current is supplied to the jaw 17. In this state, the handpiece 1 of the ultrasonic therapeutic apparatus is driven in the bipolar mode in which the probe 11 and jaw 17 are used as high-frequency electrodes. At this time, by outputting ultrasonic as energy from the probe 11 at the same time, strong coagulation or quick incision can be performed on the living body tissue between the probe distal end 11a and the jaw 17.

Therefore, in the handpiece 1 of the ultrasonic therapeutic apparatus of the present embodiment, even with the use of the single handpiece 1, selective switching can easily be effected between the bipolar mode and the probe-only output mode in accordance with the operation of the movable handle 49, for example, depending on the phase in use, and proper therapeutic treatment can be performed. Thus, there is no need to perform such a time-consuming operation that an operation is continued after exchanging a monopolar therapeutic device and a bipolar therapeutic device. As a result, in the present embodiment, like the first embodiment, compared to the case of continuing an operation by exchanging the monopolar therapeutic device and bipolar therapeutic device, it is possible to improve the operability for the user and to prevent an increase in total time for therapeutic treatment in a surgical operation.

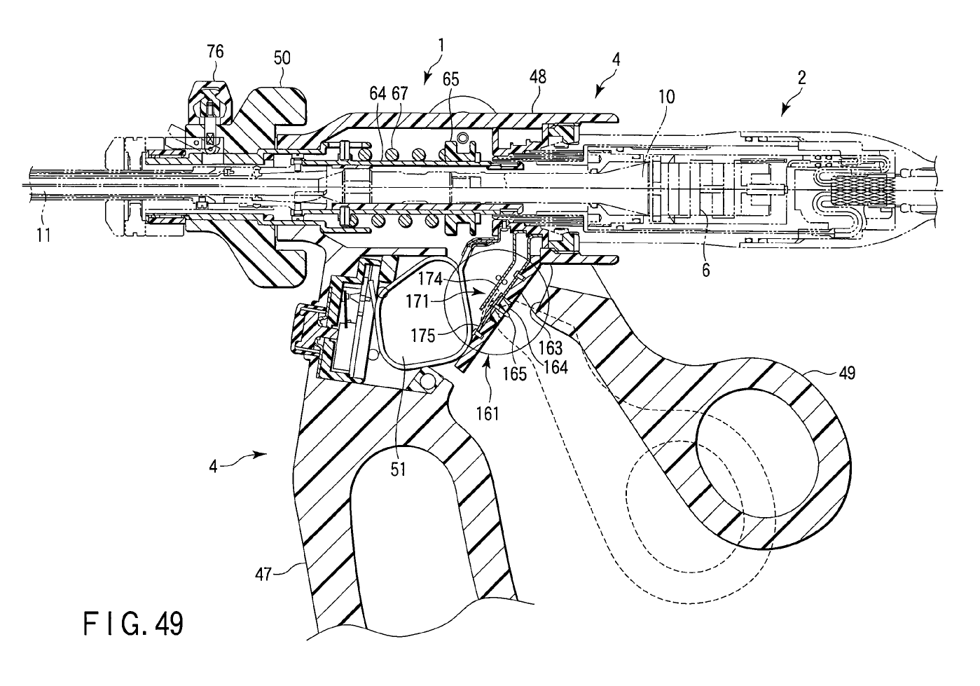

FIG. 49 to FIG. 51 show a third embodiment of the present embodiment. FIG. 49 is a longitudinal cross-sectional view showing an internal structure of a handpiece 1 of an ultrasonic therapeutic apparatus according to the present embodiment. The present embodiment is provided with an output mode switching section 161 which is configured differently from the handpiece 1 of the first embodiment (see FIG. 1 to FIG. 45). In the other structural parts, the third embodiment is substantially the same as the first embodiment (see FIG. 1 to FIG. 45). Thus, in FIG. 49 to FIG. 51, the parts common to those in the first embodiment are denoted by like reference numerals, and a description thereof is omitted.

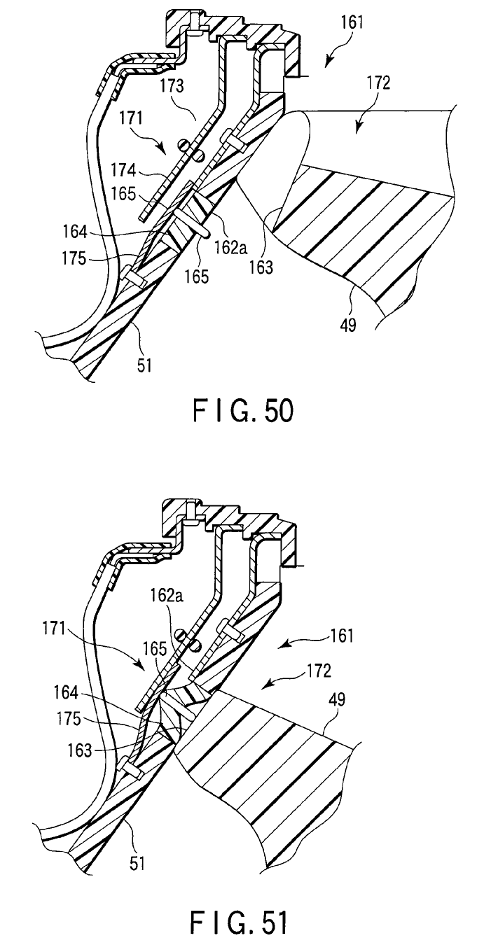

As shown in FIG. 50 and FIG. 51, the output mode switching section 161 of the present embodiment includes a contact-point switching section 171 which is mounted inside a rear-side wall portion 162 of the switch hold section 51 of the handle unit 4, and a contact-point operation section 172 which operates the contact-point switching section 171.

The contact-point switching section 171 includes a switch member 173 which is provided midway along the path of high-frequency current to the jaw 17. The switch member 173 includes a stationary contact-point member 174 and a movable contact-point member 175 which is connectable/disconnectable to/from the stationary contact-point member 174. One end portion of the movable contact-point member 175 is fixed to an inner surface of the rear-side wall portion 162 of the switch hold section 51. The other end portion of the movable contact-point member 175 is held at a position apart from the stationary contact-point member 174.

In the rear-side wall portion 162 of the switch hold section 51, an opening portion 162a is formed at a position corresponding to the other end portion of the movable contact-point member 175. An elastic deformation portion 164, such as an elastically deformable rubber plate, is provided in the opening portion 162a. A press pin 165 is projectingly provided in the elastic deformation portion 164.

The movable handle 49 is provided with a pressing portion 163 which is disposed at a coupling part between the two arms 56a and 56b and presses the press pin 165 of the elastic deformation portion 164 of the switch hold section 51. When the movable handle 49 is closed, the pressing portion 163 of the movable handle 49 presses the press pin 165, thereby pressing the other end portion of the movable contact-point member 175 in such a direction as to come in contact with the stationary contact-point member 174. Thus, the switch member 173 of the contact-point switching section 171 is operated to open/close the contact point that switches on/off the electrical conduction state of a high-frequency current to the jaw 17.

When the movable handle 49 is operated, the pressing portion 163 of the movable handle 49 moves in such a direction as to come in contact, and go out of contact, with the press pin 165. In accordance with the operation of the pressing portion 163, switching is effected between the state in which the pressing portion 163 presses the press pin 165 to put the other end portion of the movable contact-point member 175 in pressure contact with the stationary contact-point member 174, as shown in FIG. 51, and the state in which the pressing portion 163 goes out of contact with the press pin 165 to separate the other end portion of the movable contact-point member 175 from the stationary contact-point member 174, as shown in FIG. 50.

FIG. 50 shows the shift state of the output mode switching section 161 in the case where the jaw 17 is opened. In this state, the movable handle 49 is rotated in a direction away from the stationary handle 47, and the pressing portion 163 goes out of contact with the press pin 165 and the other end portion of the movable contact-point member 175 is separated from the stationary contact-point member 174. At this time, since the switch member 173 is held in the open state, the supply of high-frequency current to the jaw 17 is turned off. Thus, the output mode switching section 161 is switched to the probe-only output mode. In the probe-only output mode, only ultrasonic from the transducer 6 is output to the probe 11.

FIG. 51 shows the shift state of the output mode switching section 161 in the case where the jaw 17 is closed. In this state, the movable handle 49 is rotated in a direction toward the stationary handle 47, and the pressing portion 163 of the movable handle 49 presses the press pin 165 and the other end portion of the movable contact-point member 175 is put in pressure contact with the stationary contact-point member 174. At this time, the switch member 173 is closed, and the supply of high-frequency current to the jaw 17 is turned on. In this case, the output mode switching section 161 is switched to the bipolar mode. In the bipolar mode, a high-frequency current is supplied to the jaw 17. In this state, the handpiece 1 of the ultrasonic therapeutic apparatus is driven in the bipolar mode in which the probe 11 and jaw 17 are used as high-frequency electrodes. In this case, such a structure may be adopted that ultrasonic is output as energy from the probe 11 at the same time.

With the above-described structure, the following advantageous effects can be obtained. Specifically, the handpiece 1 of the ultrasonic therapeutic apparatus of the present embodiment is provided with the output mode switching section 161. In accordance with the operation of the movable handle 49, the output of the surgical operating apparatus is selectively switched between the bipolar mode in which the probe distal end 11a and jaw 17 are used as high-frequency electrodes and driven in a bipolar mode, and the probe-only output mode in which energy is output from only the probe distal end 11a. In the case where the movable handle 49 is rotated in a direction away from the stationary handle 47 (“opening operation time”), the output mode switching section 161 is switched to the probe-only output mode shown in FIG. 50. In this case, in the present embodiment, since the supply of high-frequency current to the jaw 17 is turned off, only ultrasonic from the transducer 6 is output to the probe 11. Accordingly, in this case, as shown in FIG. 45, therapeutic treatment is conducted on the patient P with only ultrasonic vibration which is output from the probe distal end 11a. Thus, for example, therapeutic treatment, such as puncture, can be performed by using cavitation of the probe 11.

In the case where the movable handle 49 is rotated in a direction toward the stationary handle 47 (“closing operation time”), the output mode switching section 161 is switched to the bipolar mode shown in FIG. 51. In this case, a high-frequency current is supplied to the jaw 17. In this state, the handpiece 1 of the ultrasonic therapeutic apparatus is driven in the bipolar mode in which the probe 11 and jaw 17 are used as high-frequency electrodes. At this time, by outputting ultrasonic as energy from the probe 11 at the same time, strong coagulation or quick incision can be performed on the living body tissue between the probe distal end 11a and the jaw 17.

Therefore, in the handpiece 1 of the ultrasonic therapeutic apparatus of the present embodiment, selective switching can easily be effected between the bipolar mode and the probe-only output mode in accordance with the operation of the movable handle 49, and proper therapeutic treatment can be performed. Thus, there is no need to perform such a time-consuming operation that an operation is continued after exchanging a monopolar therapeutic device and a bipolar therapeutic device. As a result, in the present embodiment, like the first embodiment, compared to the case of continuing an operation by exchanging the monopolar therapeutic device and bipolar therapeutic device, it is possible to improve the operability for the user and to prevent an increase in total time for therapeutic treatment in a surgical operation.

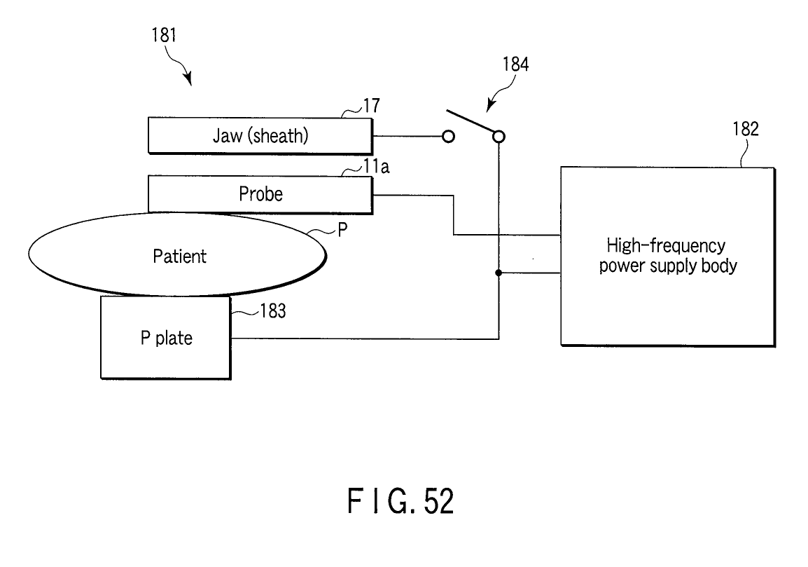

FIG. 52 shows a fourth embodiment of the present embodiment. FIG. 52 schematically shows the structure of an electric circuit of the entire system of an ultrasonic therapeutic apparatus according to the present embodiment. The present embodiment is provided with a surgical operating apparatus 181 which is configured differently from the system of the ultrasonic therapeutic apparatus of the first embodiment (see FIG. 1 to FIG. 45). In the other structural parts, the fourth embodiment is substantially the same as the first embodiment (see FIG. 1 to FIG. 45). Thus, in FIG. 52, the parts common to those in the first embodiment are denoted by like reference numerals, and a description thereof is omitted.

The surgical operating apparatus 181 of the present embodiment is a system in which the handpiece 1 of the ultrasonic therapeutic apparatus of the first embodiment (see FIG. 1 to FIG. 45) is used as a high-frequency therapeutic apparatus. The handpiece 1 of the present embodiment is connected to a high-frequency power supply body 182. A P plate 183, which is a counter-electrode plate for a monopolar therapeutic device, is connected to the high-frequency power supply body 182. The P plate 183 is set on a bed for therapeutic treatment, on which the patient P is placed. In the case where the probe distal end 11a of the probe 11 of the handpiece 1 is used as a monopolar therapeutic device, the patient P is interposed between the probe distal end 11a of the probe 11 and the P plate 183.

In the system of the present embodiment, there is provided an output mode switching section 184 which is a switch for switching the output of the surgical operating apparatus between a bipolar mode and a probe-only output mode. The output mode switching section 184 switches the output of the surgical operating apparatus to the bipolar mode, in the case where a living body tissue is held between the probe distal end 11a and the jaw 17 in accordance with the operation of the movable handle 49 and the jaw 17 is substantially closed relative to the probe distal end 11a. In this case, high-frequency current is supplied to the probe distal end 11a and the jaw 17, respectively. In this state, the handpiece 1 of the ultrasonic operating apparatus is driven in the bipolar mode in which the probe 11 and jaw 17 are used as high-frequency electrodes. Thereby, the living body tissue is clamped between the probe distal end 11a and the jaw 17, and strong coagulation can be performed on the living body tissue.

The output mode switching section 184 switches the output of the surgical operating apparatus to the probe-only output mode, in the case where the jaw 17 is not substantially closed relative to the probe distal end 11a (the open state of the jaw 17). In the system of the present embodiment, in the probe-only output mode, only high-frequency current is output as energy from the probe distal end 11a of the probe 11. In this case, the handpiece 1 is used as a monopolar therapeutic device. Monopolar therapeutic treatment is performed in the state of use in which the patient P is interposed between the probe distal end 11a of the probe 11 and the P plate 183. Thereby, quick incision treatment can be performed without the living body tissue being clamped by the probe distal end 11a.

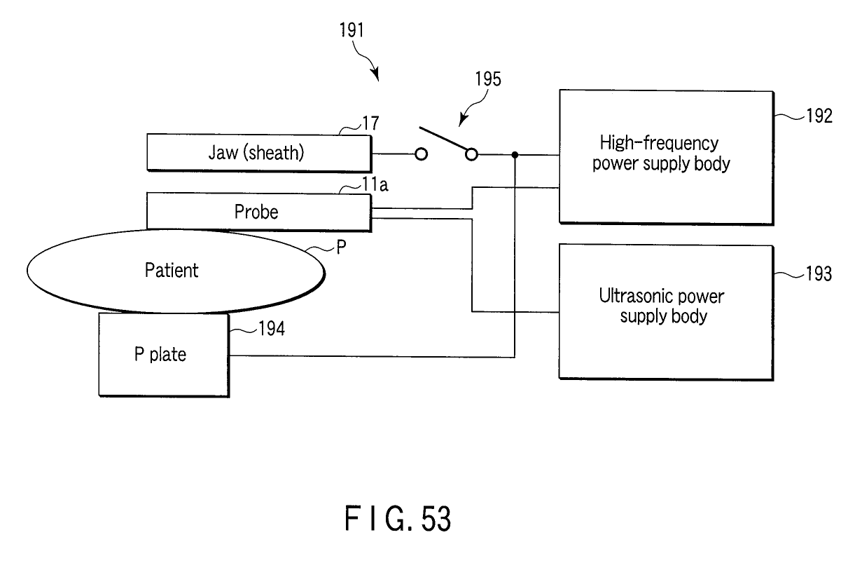

FIG. 53 shows a fifth embodiment of the present embodiment. FIG. 53 schematically shows the structure of an electric circuit of the entire system of an ultrasonic therapeutic apparatus according to the present embodiment. The present embodiment is provided with a surgical operating apparatus 191 which is configured differently from the system of the ultrasonic therapeutic apparatus of the first embodiment (see FIG. 1 to FIG. 45). In the other structural parts, the fifth embodiment is substantially the same as the first embodiment (see FIG. 1 to FIG. 45). Thus, in FIG. 53, the parts common to those in the first embodiment are denoted by like reference numerals, and a description thereof is omitted.

The surgical operating apparatus 191 of the present embodiment is a system in which the handpiece 1 of the ultrasonic therapeutic apparatus of the first embodiment (see FIG. 1 to FIG. 45) is used as a composite-type therapeutic apparatus in which high-frequency waves and ultrasonic are combined. The handpiece 1 of the present embodiment is connected to a high-frequency power supply body 192 and an ultrasonic power supply body 193. A P plate 194, which is a counter-electrode plate for a monopolar therapeutic device, is connected to the high-frequency power supply body 192. The P plate 194 is set on a bed for therapeutic treatment, on which the patient P is placed. In the case where the probe distal end 11a of the probe 11 of the handpiece 1 is used as a monopolar therapeutic device, the patient P is interposed between the probe distal end 11a of the probe 11 and the P plate 194.

In the system of the present embodiment, there is provided an output mode switching section 195 which is a switch for switching the output of the surgical operating apparatus between two modes (a first mode and a second mode). The output mode switching section 195 switches the output of the surgical operating apparatus to the first mode, in the case where a living body tissue is held between the probe distal end 11a and the jaw 17 in accordance with the operation of the movable handle 49 and the jaw 17 is substantially closed relative to the probe distal end 11a.

The first mode is a mode in which the high-frequency therapeutic device of the surgical operating apparatus is driven in the bipolar mode and at the same time the ultrasonic transducer 6 is driven. In this case, a high-frequency current is supplied to the probe distal end 11a and the jaw 17, respectively. At the same time, ultrasonic vibration is transmitted to the probe distal end 11a. In this state, the handpiece 1 of the surgical operating apparatus is driven in the bipolar mode in which the probe 11 and jaw 17 are used as high-frequency electrodes. Thereby, the living body tissue is clamped between the probe distal end 11a and the jaw 17, and strong coagulation and quick incision can be performed on the living body tissue.

The output mode switching section 195 effects switching to the second mode, in the case where the jaw 17 is not substantially closed relative to the probe distal end 11a (the open state of the jaw 17).

In the second mode, the output of the surgical operating apparatus is switched to the probe-only output mode. In the system of the present embodiment, in the probe-only output mode, ultrasonic vibration is transmitted as energy from the probe distal end 11a of the probe 11, and a high-frequency current is output. In this case, the handpiece 1 is used as a monopolar therapeutic device. Monopolar therapeutic treatment is performed in the state in which the patient P is interposed between the probe distal end 11a of the probe 11 and the P plate 194. In this case, at the same time, ultrasonic vibration is transmitted as energy from the probe distal end 11a of the probe 11. Thereby, quick incision treatment can be performed without the living body tissue being clamped by the probe distal end 11a. At this time, incision and puncture in the living body tissue can be performed, without the living body tissue adhering to the probe distal end 11a.

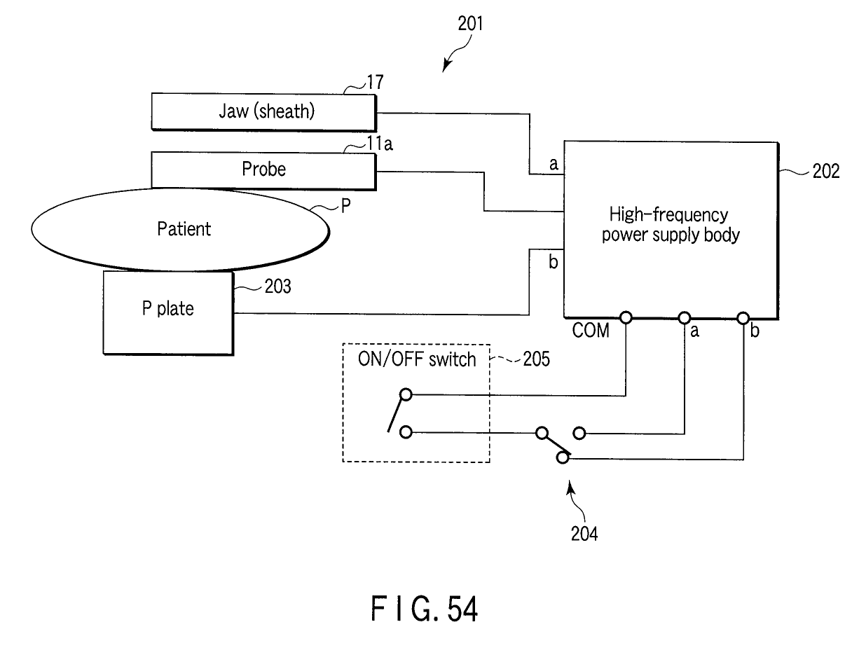

FIG. 54 shows a sixth embodiment of the present embodiment. FIG. 54 schematically shows the structure of an electric circuit of the entire system of an ultrasonic therapeutic apparatus according to the present embodiment. The present embodiment is provided with a surgical operating apparatus 201 which is configured differently from the system of the ultrasonic therapeutic apparatus of the first embodiment (see FIG. 1 to FIG. 45). In the other structural parts, the sixth embodiment is substantially the same as the first embodiment (see FIG. 1 to FIG. 45). Thus, in FIG. 54, the parts common to those in the first embodiment are denoted by like reference numerals, and a description thereof is omitted.

The surgical operating apparatus 201 of the present embodiment is a system in which the handpiece 1 of the ultrasonic therapeutic apparatus of the first embodiment (see FIG. 1 to FIG. 45) is used as a high-frequency therapeutic apparatus. The handpiece 1 of the present embodiment is connected to a high-frequency power supply body 202. A P plate 203, which is a counter-electrode plate for a monopolar therapeutic device, is connected to the high-frequency power supply body 202. The P plate 203 is set in the state in which the patient P is interposed between the probe distal end 11a of the probe 11 and the P plate 203, in the case where the probe distal end 11a of the probe 11 of the handpiece 1 is used as a monopolar therapeutic device.

In the system of the present embodiment, there is provided an output mode switching section 204 which is a switch for switching the output of the surgical operating apparatus between a bipolar mode and a probe-only output mode. In addition, an ON/OFF switch 205 is connected between the output mode switching section 204 and the high-frequency power supply body 202.

The output mode switching section 204 switches the output of the surgical operating apparatus to the bipolar mode, in the case where a living body tissue is held between the probe distal end 11a and the jaw 17 in accordance with the operation of the movable handle 49 and the jaw 17 is substantially closed relative to the probe distal end 11a. In this case, the electric circuit is changed over to an (a) terminal side. Thus, a high-frequency current is supplied to the probe distal end 11a and the jaw 17, respectively. In this state, the handpiece 1 of the surgical operating apparatus is driven in the bipolar mode in which the probe 11 and jaw 17 are used as high-frequency electrodes. Thereby, the living body tissue is clamped between the probe distal end 11a and the jaw 17, and strong coagulation can be performed on the living body tissue. In the meantime, during the above-described operation, the supply of high-frequency current can be turned off by the operation of the ON/OFF switch 205.

The output mode switching section 204 switches the output of the surgical operating apparatus to the probe-only output mode, in the case where the jaw 17 is not substantially closed relative to the probe distal end 11a (the open state of the jaw 17). In the system of the present embodiment, in the probe-only output mode, only high-frequency current is output as energy from the probe distal end 11a of the probe 11. In this case, the electric circuit is changed over to a (b) terminal side. Thus, the handpiece 1 is used as a monopolar therapeutic device. Monopolar therapeutic treatment is performed in the state of use in which the patient P is interposed between the probe distal end 11a of the probe 11 and the P plate 203. Thereby, quick incision treatment can be performed without the living body tissue being clamped by the probe distal end 11a. In the meantime, during the above-described operation, the supply of high-frequency current can be turned off by the operation of the ON/OFF switch 205.

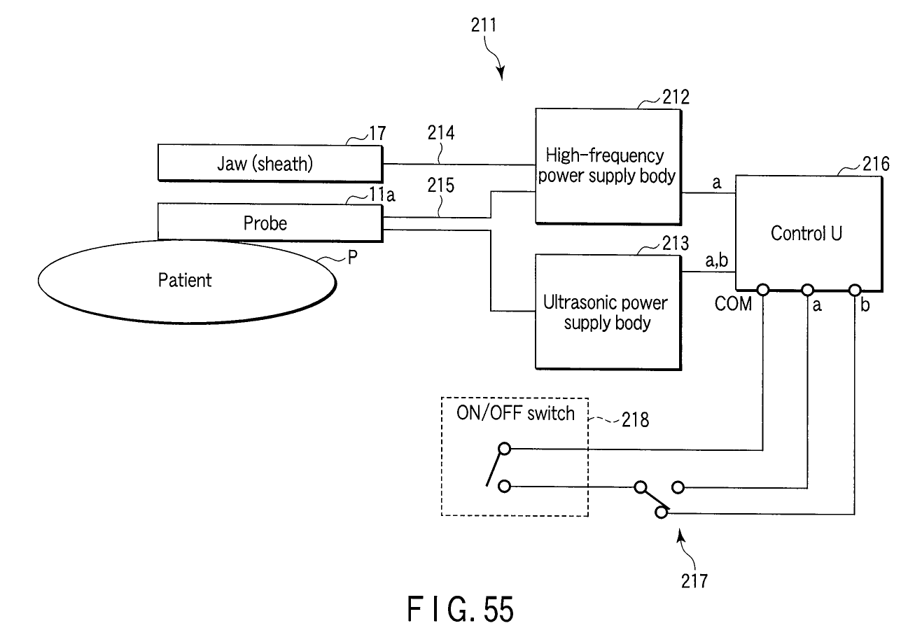

FIG. 55 shows a seventh embodiment of the present embodiment. FIG. 55 schematically shows the structure of an electric circuit of the entire system of an ultrasonic therapeutic apparatus according to the present embodiment. The present embodiment is provided with a surgical operating apparatus 211 which is configured differently from the system of the ultrasonic therapeutic apparatus of the first embodiment (see FIG. 1 to FIG. 45). In the other structural parts, the seventh embodiment is substantially the same as the first embodiment (see FIG. 1 to FIG. 45). Thus, in FIG. 55, the parts common to those in the first embodiment are denoted by like reference numerals, and a description thereof is omitted.

The surgical operating apparatus 211 of the present embodiment is a system in which the handpiece 1 of the ultrasonic therapeutic apparatus of the first embodiment (see FIG. 1 to FIG. 45) is used as a composite-type therapeutic apparatus in which high-frequency waves and ultrasonic are combined. The handpiece 1 of the present embodiment is connected to a high-frequency power supply body 212 and an ultrasonic power supply body 213. A first electric path 214, which is connected to the jaw 17, and a second electric path 215, which is connected to the probe distal end 11a, are connected to the high-frequency power supply body 212. In addition, the ultrasonic transducer 6 of the handpiece 1 is connected to the ultrasonic power supply body 213.

In the system of the present embodiment, the high-frequency power supply body 212 and the ultrasonic power supply body 213 are connected to a control unit 216. An output mode switching section 217, which is a switch for switching the output of the surgical operating apparatus between two modes (a first mode and a second mode), is connected to the control unit 216. In addition, an ON/OFF switch 218 is connected between the output mode switching section 217 and the control unit 216.

The output mode switching section 217 switches the output of the surgical operating apparatus to the first mode, in the case where a living body tissue is held between the probe distal end 11a and the jaw 17 in accordance with the operation of the movable handle 49 and the jaw 17 is substantially closed relative to the probe distal end 11a.

The first mode is a mode in which the high-frequency therapeutic device of the surgical operating apparatus is driven in the bipolar mode and at the same time the ultrasonic transducer 6 is driven. In this case, the electric circuit is changed over to an (a) terminal side. Thus, a high-frequency current is supplied to the probe distal end 11a and the jaw 17, respectively. At the same time, ultrasonic vibration is transmitted to the probe distal end 11a. In this state, the handpiece 1 of the surgical operating apparatus is driven in the bipolar mode in which the probe 11 and jaw 17 are used as high-frequency electrodes. Thereby, the living body tissue is clamped between the probe distal end 11a and the jaw 17, and strong coagulation and quick incision can be performed on the living body tissue. In the meantime, during the above-described operation, the supply of current can be turned off by the operation of the ON/OFF switch 218.

The output mode switching section 217 effects switching to the second mode, in the case where the jaw 17 is not substantially closed relative to the probe distal end 11a (the open state of the jaw 17).

In the second mode, the output of the surgical operating apparatus is switched to the probe-only output mode. In this case, the electric circuit is changed over to a (b) terminal side. Thus, in the system of the present embodiment, in the probe-only output mode, only ultrasonic vibration is transmitted as energy from the probe distal end 11a of the probe 11. Thereby, for example, therapeutic treatment, such as puncture, can be performed by using cavitation of the probe 11. In the meantime, during the above-described operation, the supply of current can be turned off by the operation of the ON/OFF switch 218.

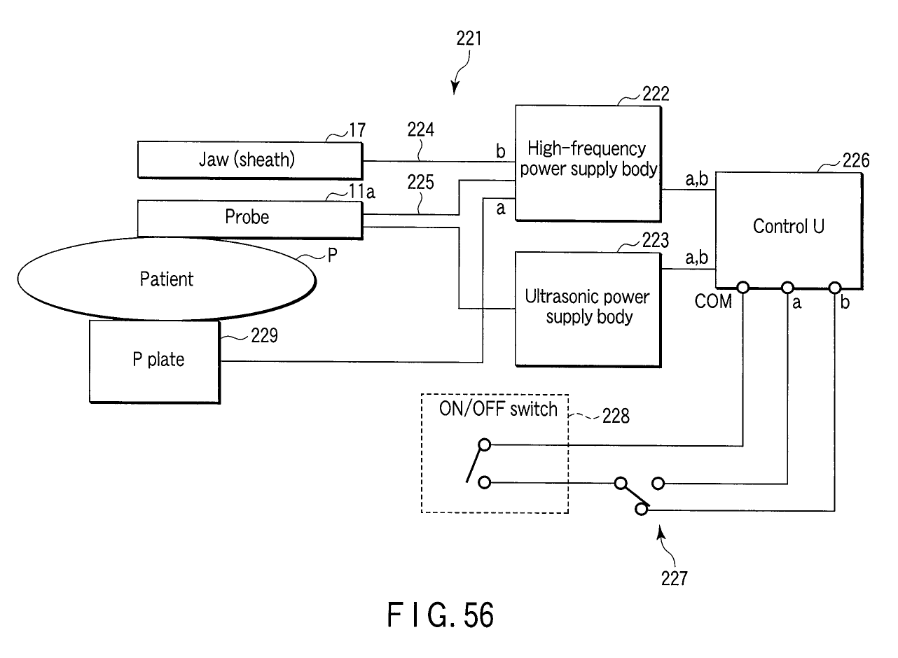

FIG. 56 shows an eighth embodiment of the present embodiment. FIG. 56 schematically shows the structure of an electric circuit of the entire system of an ultrasonic therapeutic apparatus according to the present embodiment. The present embodiment is provided with a surgical operating apparatus 221 which is configured differently from the system of the ultrasonic therapeutic apparatus of the first embodiment (see FIG. 1 to FIG. 45). In the other structural parts, the eighth embodiment is substantially the same as the first embodiment (see FIG. 1 to FIG. 45). Thus, in FIG. 56, the parts common to those in the first embodiment are denoted by like reference numerals, and a description thereof is omitted.

The surgical operating apparatus 221 of the present embodiment is a system in which the handpiece 1 of the ultrasonic therapeutic apparatus of the first embodiment (see FIG. 1 to FIG. 45) is used as a composite-type therapeutic apparatus in which high-frequency waves and ultrasonic are combined. The handpiece 1 of the present embodiment is connected to a high-frequency power supply body 222 and an ultrasonic power supply body 223. A first electric path 224, which is connected to the jaw 17, and a second electric path 225, which is connected to the probe distal end 11a, and a P plate 229, which is a counter-electrode plate for a monopolar therapeutic device, are connected to the high-frequency power supply body 222. In addition, the ultrasonic transducer 6 of the handpiece 1 is connected to the ultrasonic power supply body 223.

In the system of the present embodiment, the high-frequency power supply body 222 and the ultrasonic power supply body 223 are connected to a control unit 226. An output mode switching section 227, which is a switch for switching the output of the surgical operating apparatus between two modes (a first mode and a second mode), is connected to the control unit 226. In addition, an ON/OFF switch 228 is connected between the output mode switching section 227 and the control unit 226.

The output mode switching section 227 switches the output of the surgical operating apparatus to the first mode, in the case where a living body tissue is held between the probe distal end 11a and the jaw 17 in accordance with the operation of the movable handle 49 and the jaw 17 is substantially closed relative to the probe distal end 11a.

The first mode is a mode in which the high-frequency therapeutic device of the surgical operating apparatus is driven in the bipolar mode and at the same time the ultrasonic transducer 6 is driven. In this case, the electric circuit is changed over to an (a) terminal side. Thus, a high-frequency current is supplied to the probe distal end 11a and the jaw 17, respectively. At the same time, ultrasonic vibration is transmitted to the probe distal end 11a. In this state, the handpiece 1 of the surgical operating apparatus is driven in the bipolar mode in which the probe 11 and jaw 17 are used as high-frequency electrodes. Thereby, the living body tissue is clamped between the probe distal end 11a and the jaw 17, and strong coagulation and quick incision can be performed on the living body tissue. In the meantime, during the above-described operation, the supply of current can be turned off by the operation of the ON/OFF switch 228.

The output mode switching section 227 effects switching to the second mode, in the case where the jaw 17 is not substantially closed relative to the probe distal end 11a (the open state of the jaw 17).

In the second mode, the output of the surgical operating apparatus is switched to the probe-only output mode. In this case, the electric circuit is changed over to a (b) terminal side. Thus, in the system of the present embodiment, in the probe-only output mode, ultrasonic vibration is transmitted as energy from the probe distal end 11a of the probe 11, and a high-frequency current is output. In this case, the handpiece 1 is used as a monopolar therapeutic device. Monopolar therapeutic treatment is performed in the state in which the patient P is interposed between the probe distal end 11a of the probe 11 and the P plate 229. In this case, at the same time, ultrasonic vibration is transmitted as energy from the probe distal end 11a of the probe 11. Thereby, quick incision treatment can be performed without the living body tissue being clamped by the probe distal end 11a. At this time, incision and puncture in the living body tissue can be performed, without the living body tissue adhering to the probe distal end 11a. In the meantime, during the above-described operation, the supply of current can be turned off by the operation of the ON/OFF switch 228.

Needless to say, the present invention is not limited directly to the above-described embodiments, and various modifications may be made without departing from the spirit of the invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A surgical operating apparatus comprising:
a probe for outputting energy for performing therapeutic treatment of a living body;
a jaw which is openable/closable relative to the probe; and
an output mode switching section which selectively switches, in accordance with an open/closed state of the jaw, an output of the surgical operating apparatus between a bipolar mode in which the probe and the jaw are used as high-frequency electrodes, and a probe-only output mode in which the energy is output from only the probe.

2. The surgical operating apparatus according to claim 1, wherein the surgical operating apparatus further includes an operation section for opening/closing the jaw, and
the output mode switching section switches the output of the surgical operating apparatus to the bipolar mode in a case where a living body tissue is held between the probe and the jaw in accordance with an operation of the operation section and the jaw is substantially closed relative to the probe, and switches the output of the surgical operating apparatus to the probe-only output mode in a case where the jaw is in an open state in which the jaw is not substantially closed relative to the probe.

3. The surgical operating apparatus according to claim 1, wherein in the probe-only output mode, only a high-frequency current is output as the energy from the probe.

4. The surgical operating apparatus according to claim 1, wherein the surgical operating apparatus further includes an ultrasonic output section which supplies an ultrasonic output to the probe.

5. The surgical operating apparatus according to claim 4, wherein in the probe-only output mode, only ultrasonic from the ultrasonic output section is output as the energy from the probe.

6. The surgical operating apparatus according to claim 4, wherein in the probe-only output mode, a high-frequency wave and ultrasonic are simultaneously output as the energy from the probe.

7. The surgical operating apparatus according to claim 4, wherein in the bipolar mode, ultrasonic is also output as the energy from the probe at the same time.

8. A surgical operating apparatus comprising:
a probe for outputting energy for performing therapeutic treatment of a living body;
a jaw which is openable/closable relative to the probe;
an operation section for opening/closing the jaw; and
an output mode switching section which selectively switches an output mode of the probe between a probe-only output mode in which the energy is output from only the probe, and a bipolar mode in which the probe and the jaw are used as high-frequency electrodes,
wherein the output mode switching section includes a contact-point switching section which opens/closes a contact point that switches on/off an electrical conduction state of a high-frequency current to the jaw in accordance with an operation of a movable member which moves in accordance with an operation of the operation section.

9. The surgical operating apparatus according to claim 8, wherein the contact-point switching section closes the contact point in accordance with the operation of the movable member at a time when the operation section moves the jaw in a direction of closing, thereby effecting switching to a state in which the high-frequency current to the jaw is rendered conductive, and opens the contact point in accordance with the operation of the movable member at a time when the operation section moves the jaw in a direction of opening, thereby effecting switching to a state in which the high-frequency current to the jaw is rendered non-conductive.

10. The surgical operating apparatus according to claim 8, wherein the movable member includes an operation force transmission member which is advanced/retreated in an axial direction of the probe in accordance with the operation of the operation section and transmits an operation force of the operation section to the jaw, and
the contact-point switching section opens/closes the contact point in accordance with an advancing/retreating operation of the operation force transmission member.

11. The surgical operating apparatus according to claim 10, wherein the contact-point switching section closes the contact point in accordance with the advancing/retreating operation of the operation force transmission member at the time when the operation section moves the jaw in a direction of closing, thereby effecting switching to a state in which the high-frequency current to the jaw is rendered conductive, and opens the contact point in accordance with the advancing/retreating operation of the operation force transmission member at the time when the operation section moves the jaw in a direction of opening, thereby effecting switching to a state in which the high-frequency current to the jaw is rendered non-conductive.

12. The surgical operating apparatus according to claim 8, wherein the operation section includes a stationary handle and a movable handle which moves in a direction toward/away from the stationary handle,
the movable member is composed of the movable handle, and
the contact-point switching section which closes the contact point in accordance with an operation of the movable handle at a time when the operation section moves the jaw in a direction of closing, thereby effecting switching to a state in which the high-frequency current to the jaw is rendered conductive, and opens the contact point in accordance with the operation of the movable handle at a time when the operation section moves the jaw in a direction of opening, thereby effecting switching to a state in which the high-frequency current to the jaw is rendered non-conductive.

13. The surgical operating apparatus according to claim 8, wherein in the probe-only output mode, only a high-frequency current is output as the energy from the probe.

14. The surgical operating apparatus according to claim 8, wherein the surgical operating apparatus further includes an ultrasonic output section which supplies an ultrasonic output to the probe.

15. The surgical operating apparatus according to claim 14, wherein in the probe-only output mode, only ultrasonic from the ultrasonic output section is output as the energy from the probe.

16. The surgical operating apparatus according to claim 14, wherein in the probe-only output mode, a high-frequency wave and ultrasonic are simultaneously output as the energy from the probe.

17. The surgical operating apparatus according to claim 14, wherein in the bipolar mode, ultrasonic is also output as the energy from the probe at the same time.

18. A surgical operating apparatus comprising:
a probe for outputting energy for performing therapeutic treatment of a living body;
a jaw which is openable/closable relative to the probe; and
output mode switching means for effecting switching between a bipolar mode in which the probe and the jaw are used as electrodes, and a probe-only output mode in which energy is output from only the probe,
wherein the output mode switching means operates in interlock with an opening/closing operation of the jaw, effects switching to the bipolar mode at a time of a closing operation of the jaw, and effects switching to the probe-only output mode at a time of a openable operation of the jaw.
